(19) United States
(12) Patent Application Publication
(10) Pub. No.: US 2006/0099587 A1
(43) Pub. Date: May 11, 2006

(54) Alphavirus vectors having attentuated virion structural proteins

(76) Inventors: Robert E Johnston, Chapel Hill, NC (US); Nancy L Davis, Chapel Hill, NC (US); Ande West, Carrboro, NC (US); Jonathan Smith, Cary, NC (US)

Correspondence Address: MYERS BIGEL SIBLEY & SAJOVEC, PO BOX 37428, RALEIGH, NC, 27627, US

(21) Appl. No.: 10/517,083

(22) PCT Filed: Jun. 20, 2003
(86) PCT No.: PCT/US03/19626

§ 371 Date: Sep. 29, 2005

(51) Int. Cl. C12Q 1/68; C12N 7/00; C12N 7/01

(52) U.S. Cl. 435/6; 435/235.1

(57) ABSTRACT

The present invention provides immunogenic compositions and methods that may be used to administer safer (i.e., attenuated) alphavirus vectors (such as alphavirus vectors comprising a VEE virion shell) that retain improved immunogenicity as compared with other attenuated alphaviruses (e.g., the VEE 3014 mutant, described below). In particular embodiments of the invention, the alphavirus vector comprises VEE structural proteins comprising an attenuating mutation in the E1 glycoprotein. In other particular embodiments, the attenuating mutation is in the fusogenic region of the E1 glycoprotein. The present invention enables administration of lower dosages of a safer (i.e., attenuated) virus and, thus, can further reduce manufacturing costs. The present inventors have found that immunogenicity of alphavirus vectors may be influenced by a number of factors including species, site and route of administration.

### RELATED APPLICATION INFORMATION

This application claims the benefit of U.S. Provisional Application No. 60/390,774, Filed 21 Jun. 2002, the disclosure of which is incorporated herein by reference in its entirety.

### GOVERNMENT SUPPORT

The present invention was made with government support under grant numbers 5P01 A146023 and 5R01 Al51990 from the National Institutes of Health. The United States Government has certain rights to this invention.

### FIELD OF THE INVENTION

The present invention provides improved immunogenic compositions, in particular, improved immunogenic compositions comprising attenuated alphavirus virion shells and methods of administering the same in vitro and in vivo.

### BACKGROUND OF THE INVENTION

Venezuelan Equine Encephalitis virus (VEE) is a positive-sense RNA virus responsible for the mosquito-borne epidemic encephalomyelitis in humans and a wide variety of equids in tropical and sub-tropical areas of the New World. Initial studies to develop a vaccine against encephalytic disease lead to the development of an attenuated, live virus vaccine by introducing a variety of attenuating mutations into the virulent parental genome. As an outgrowth of the studies characterizing the biological consequences of these attenuating mutations, the use of replication-defective virus particles, termed viral replicon particles, has shown great promise as a viral vector delivery system. Replicons are constructed to carry one or more heterologous antigens in place of some or all of the structural genes. The replicons are introduced into target cells along with a helper construct(s) that expresses the viral structural protein(s) not encoded by the replicon or, alternatively, the replicon is introduced into a packaging cell capable of expressing the structural proteins. The replicons then express the introduced heterologous antigen(s) at very high levels from the subgenomic mRNA. Subsequent viral progeny are prevented from assembly since the replicons do not encode all of the essential viral packaging genes. Studies with the replicon system have shown great promise as vector systems as demonstrated by their ability to: (1) target to lymphoid tissue, (2) express high levels of antigen, (3) induce protective humoral, cellular and mucosal immune responses that give protection against challenge, and (4) respond to boost after a primary response (e.g., the boost is not precluded by pre-existing immunity to the vector itself).

As described above, alphavirus replicon particles have been developed with attenuating mutations so as to increase the safety of virus administration. Unfortunately, however, attenuating mutations have been associated with a decrease in potency, resulting in the need to deliver larger doses of particles carrying such attenuating mutations to obtain the desired immunological response following virus administration. Accordingly, there remains a need in the art for improved alphavirus vaccines that have the features of both safety and efficacy.

### SUMMARY OF THE INVENTION

The present invention provides immunogenic compositions and methods that may be used to administer safer (i.e., attenuated) alphavirus vectors (such as alphavirus vectors comprising a VEE virion shell) that retain improved immunogenicity as compared with attenuated alphaviruses (e.g., the VEE 3014 mutant, described below). In particular embodiments of the invention, the alphavirus vector comprises VEE structural proteins comprising an attenuating mutation in the E1 glycoprotein. The present invention enables administration of lower dosages of a safer (i.e., attenuated) virus and, thus, can further reduce manufacturing costs. The present inventors have found that immunogenicity of alphavirus vectors may be influenced by a number of factors including species, site and route of administration.

### BRIEF DESCRIPTION OF THE DRAWINGS

FIG. 1. Primary anti-HA response in mice to HA-VRP immunization. Mice were challenged with HA-VRP-3000 or HA-VRP 3014, and bled after 28 days. ELISA assays were performed as described in Example 1.

FIG. 2. Secondary anti-HA response in mice to HA-VRP immunization. At 28 days following primary inoculation, mice were boosted with a second administration of HA-VRP-3000 or HA-VRP 3014, and bled 28 days following booster administration. ELISA assays were performed as described in Example 1.

FIG. 3. CTL response to HIV Clade C gag in mice primed and boosted with HIV_{gag}-VRP-3000.

FIG. 4. Effect of VRP-replicon coat protein on CTL response in mice primed and boosted with HIV Clade C gag VRP with wild-type (VRP-3000) and mutant (VRP-3014) coat protein at an effector/target ratio of 25:1.

FIG. 5. Effect of different VRP-replicon coat proteins on immunization. Mice were inoculated with HA-VRP-3000 (wild-type), HA-VRP-3014, HA-VRP-3040, and HA-VRP3042 (mutant) as described in Example 4.

FIG. 6. Effect of mode of administration of HA-VRP on Anti-HA response. Mice were inoculated via footpad, subcutaneous, or intradermal inoculation as described in Example 5, boosted at 28 days, and bled at 28 days following booster inoculation.

FIG. 7. Targeting of dendritic cells with GFP-VRP in macaques. GFP-VRP-3000 (wild-type) was administered to rhesus macaques as described in Example 6, and inguinal lymph nodes were harvested 18 hours post-injection. Fluorescence microscopy was performed as described in Example 1.

### DETAILED DESCRIPTION OF THE PREFERRED EMBODIMENTS

The present invention addresses the need in the art for improved attenuated alphavirus vectors. The alphavirus vectors of the invention comprise attenuated virion shells or coats (e.g., a VEE coat) but retain improved immunogenicity as compared with other attenuated alphaviruses (e.g., the VEE 3014 mutant, described below). Thus, the present invention may enable administration of lower dosages of a safer (i.e., attenuated) virus and, thus, may further reduce manufacturing costs. The present invention is further based on the finding that the immunogenicity of the alphavirus may be enhanced by both the site and route of administration.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Except as otherwise indicated, standard methods known to those skilled in the art may be used for the construction and use of recombinant nucleotide sequences, vectors, helper constructs, transformed host cells, selectable markers, alphavirus vectors, viral infection of cells, production of attenuated viruses, and the like. Such techniques are known to those skilled in the art. See, e.g., SAMBROOK et al., MOLECULAR CLONING: A LABORATORY MANUAL 3rd Ed. (Cold Spring Harbor, N.Y., 2001); F. M. AUSUBEL et al. CURRENT PROTOCOLS IN MOLECULAR BIOLOGY (Green Publishing Associates, Inc. and John Wiley & Sons, Inc., New York).

I. Definitions.

The term “alphavirus” has its conventional meaning in the art, and includes Eastern Equine Encephalitis virus (EEE), Venezuelan Equine Encephalitis virus (VEE), Everglades virus, Mucambo virus, Pixuna virus, Western Encephalitis virus (WEE), Sindbis virus, including TR339, South African Arbovirus No. 86 (S.A.AR86), Girdwood S.A. virus, Ockelbo virus, Semliki Forest virus, Middelburg virus, Chikungunya virus, O'Nyong-Nyong virus, Ross River virus, Barmah Forest virus, Getah virus, Sagiyama virus, Bebaru virus, Mayaro virus, Una virus, Aura virus, Whataroa virus, Babanki virus, Kyzlagach virus, Highlands J virus, Fort Morgan virus, Ndumu virus, Buggy Creek virus, and any other virus classified by the International Committee on Taxonomy of Viruses (ICTV) as an alphavirus.

In particular embodiments of the invention, the alphavirus has a VEE virion shell. According to this embodiment, the alphavirus may be a chimeric alphavirus and have a genomic RNA from another alphavirus. Alternatively, the alphavirus virion comprises a VEE E1 glycoprotein and may comprise structural proteins (e.g., capsid and/or E2 glycoprotein) from other alphaviruses. In other embodiments, the alphavirus is a VEE virus having both a VEE coat and genomic RNA.

An “Old World alphavirus” is a virus that is primarily distributed throughout the Old. World. Alternately stated, an Old World alphavirus is a virus that is primarily distributed throughout Africa, Asia, Australia and New Zealand, or Europe. Exemplary Old World viruses include SF group alphaviruses and SIN group alphaviruses. SF group alphaviruses include Semliki Forest virus, Middelburg virus, Chikungunya virus, O'Nyong-Nyong virus, Ross River virus, Barmah Forest virus, Getah virus, Sagiyama virus, Bebaru virus, Mayaro virus, and Una virus. SIN group alphaviruses include Sindbis virus, South African Arbovirus No. 86, Ockelbo virus, Girdwood S.A. virus, Aura virus, Whataroa virus, Babanki virus, and Kyzylagach virus.

The complete genomic sequences, as well as the sequences of the various structural and non-structural proteins are known in the art for numerous alphaviruses and include: Sindbis virus genomic sequence (GenBank Accession Nos. J02363, NCBI Accession No. NC_{—}001547), S.A.AR86 genomic sequence (GenBank Accession No. U38305), VEE genomic sequence (GenBank Accession No. L04653, NCBI Accession No. NC_{—}001449), Girdwood S.A genomic sequence (GenBank Accession No. U38304), Semliki Forest virus genomic sequence (GenBank Accession No. X04129, NCBI Accession No. NC_{—}003215), and the TR339 genomic sequence (Klimstra et al., (1988) J. Virol. 72:7357; McKnight et al., (1996) J. Virol. 70:1981); the disclosures of which are incorporated herein by reference in their entireties.

The phrase “alphavirus structural protein(s)” or “VEE structural protein(s)” as used herein refers to one or more of the proteins that are required to produce a functional alphavirus/VEE virion shell. The alphavirus/VEE structural proteins include the capsid protein, E1 glycoprotein, E2 glycoprotein, E3 protein and 6K protein. As used herein, the term alphavirus “virion shell” is intended to refer to the alphavirus capsid and E1 and E2 glycoproteins assembled to form an enveloped nucleocapsid-like structure. The E3 and 6K alphavirus proteins are processed out of the mature virus. As described in more detail below, certain attenuating mutations are known to affect this processing. As previously described, the alphavirus capsid protein associates with itself and with the RNA genome to form the icosahedral nucleocapsid, which is then surrounded by a lipid envelope covered with a regular array of transmembranal protein spikes, each of which consists of a heterodimeric complex of the two alphavirus glycoproteins, E1 and E2 (See Paredes et al:, (1993) Proc. Nat. Acad. Sci. USA 90, 9095-99; Paredes et al., (1993) Virology 187, 324-32; Pedersen et al., ( 1974) J. Virol. 14:40).

An alphavirus or VEE “genomic RNA” indicates the alphavirus/VEE RNA transcript. The wild-type alphavirus genome is a single-stranded, messenger-sense RNA, modified at the 5′-end with a methylated cap, and at the 3′-end with a variable-length poly (A) tract. The viral genome is divided into two regions: the first encodes the nonstructural or replicase proteins (nsP1-nsP4) and the second encodes the viral structural proteins (Strauss and Strauss, Microbiological Rev. (1994) 58:491-562). As used herein, the term “genomic RNA” encompasses recombinant alphavirus genomes (e.g., containing a heterologous nucleotide sequence(s)), viral genomes containing one or more attenuating mutations, deletions, insertions, and/or otherwise modified viral genomes. For example, the “genomic RNA” may be modified to form a double-promoter molecule or a replicon (each as described below).

A “chimeric” alphavirus as used herein comprises an alphavirus virion shell from one alphavirus and a genomic RNA from another alphavirus. In embodiments of the invention, the chimeric alphavirus comprises VEE structural proteins. In other particular embodiments, the alphavirus comprises the VEE E1 glycoprotein.

An “infectious” alphavirus or VEE particle is one that can introduce the alphavirus/VEE genomic RNA into a permissive cell, typically by viral transduction. Upon introduction into the target cell, the genomic RNA serves as a template for RNA transcription (i.e., gene expression). The “infectious” alphavirus particle may be “replication-competent” (i.e., can transcribe and replicate the alphavirus genomic RNA) and “propagation-competent” (i.e., results in a productive infection in which new alphavirus particles are produced). In embodiments of the invention, the “infectious” alphavirus particle is a replicon particle (as described below) that can introduce the genomic RNA (i.e., replicon) into a host cell, is “replication-competent” to replicate the genomic RNA, but is “propagation-defective” in that it is unable to produce new alphavirus particles in the absence of helper sequences or a packaging cell that complements the deletions or other mutations in the replicon (i.e., provide the structural proteins that are not provided by the replicon).

As used herein, the term “polypeptide” encompasses both peptides and proteins.

As used herein, an “isolated” nucleic acid (e.g., an “isolated DNA” or an “isolated genomic RNA”) means a nucleic acid separated or substantially free from at least some of the other components of the naturally occurring organism or virus, for example, the cell or viral structural components or other polypeptides or nucleic acids commonly found associated with the nucleic acid.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

As used herein, the terms “deleted” or “deletion” mean either total deletion of the specified segment or the deletion of a sufficient portion of the specified segment to render the segment inoperative or nonfunctional (e.g., does not encode a function protein), in accordance with standard usage; See, e.g., U.S. Pat. No. 4,650,764 to Temin et al.).

The phrases “attenuating mutation” and “attenuating amino acid” as used herein mean a nucleotide mutation or an amino acid encoded in view of such mutation which result in a decreased probability of causing disease in its host (i.e., a loss of virulence), in accordance with standard terminology in the art (See, e.g., B. Davis et al., Microbiology, 132 (3d ed. 1980), whether the mutation be a substitution mutation or an in-frame deletion or insertion mutation. Attenuating mutations may be in the coding or non-coding regions of the alphavirus genome. As known by those skilled in the art, the phrase “attenuating mutation” excludes mutations or combinations of mutations which would be lethal to the virus. Those skilled in the art will appreciate that some attenuating mutations may be lethal in the absence of a second-site suppressor mutation(s).

II. Alphavirus Vectors.

The present invention is practiced using alphavirus vectors, preferably a propagation-incompetent alphavirus vector, more preferably an alphavirus replicon vector (as described below). Alphavirus and replicon vectors are described in U.S. Pat. No. 5,505,947 to Johnston et al.; U.S. Pat. No. 5,792,462 to Johnston et al., U.S. Pat. No. 5,814,482 to Dubensky et al., U.S. Pat. No. 5,843,723 to Dubensky et al., U.S. Pat. No. 5,789,245 to Dubensky et al., U.S. Pat. No. 5,739,026 to Garoff et al., the disclosures of which are incorporated herein by reference in their entireties. Typically, the alphavirus vector comprises one or more heterologous nucleic acids. In embodiments of the invention at least one of the heterologous nucleic acids encodes an antigen.

Alphavirus vectors can be transcribed in vitro from cDNA molecules, for example, from a bacterial or viral promoter. Alternatively, they can be produced in vivo from DNA, for example, from a viral or eukaryotic promoter (see, e.g., U.S. Pat. Nos. 5,814,482 and 6,015,686; incorporated in their entireties herein by reference).

In particular embodiments of the invention, the alphavirus vector has a VEE virion shell. According to this embodiment, the alphavirus may be a chimeric alphavirus and have a genomic RNA from another alphavirus. Alternatively, the alphavirus virion comprises a VEE E1 glycoprotein and may comprise structural proteins (e.g., capsid and/or E2 glycoprotein) from other alphaviruses. In other embodiments, the alphavirus is a VEE virus with both a VEE genomic RNA and virion coat.

Alphavirus vectors elicit a strong host response to the antigen(s) encoded by the heterologous sequence(s) in the vector. While not wishing to be held to any particular theory of the invention, it appears that alphavirus vectors induce a more balanced and comprehensive immune response (i.e., cellular and humoral immunity) than do conventional vaccination methods. Moreover, it appears that alphavirus vectors induce a strong immune response, in part, because they directly infect and replicate within dendritic cells. The resulting presentation of antigen to the immune system induces a strong immune response. The alphavirus 26S subgenomic promoter also appears to give high level of expression of a heterologous nucleic acid encoding an immunogen.

The alphavirus vector preparation may be partially or highly purified, or may be a relatively crude cell lysate or supernate from a cell culture, as known in the art.

A. Double Promoter Vectors.

In one embodiment of the invention, the alphavirus genomic RNA is a double promoter vector that is both replication and propagation competent. Double promoter vectors are described in U.S. Pat. Nos. 5,185,440, 5,505,947 and 5,639,650, the disclosures of which are incorporated in their entireties by reference. In embodiments of the invention, the alphavirus genomic RNA used to construct the double promoter vector is a VEE, Semliki Forest Virus, S.A.AR86, Girdwood S.A., TR339, Sindbis or Ockelbo genomic RNA. In embodiments of the invention, the double promoter vector contains one or more attenuating mutations in the genomic RNA. Attenuating mutations are described in more detail hereinbelow.

In particular embodiments, the double promoter vector is constructed so as to contain a second subgenomic promoter (i.e., 26S promoter) inserted 3′ to the virus RNA encoding the structural proteins. The heterologous RNA is inserted between the second subgenomic promoter, so as to be operatively associated therewith, and the 3′ UTR of the virus genome. Heterologous RNA sequences of less than about 3 kilobases, preferably those less than about 2 kilobases, and more preferably those less than about 1 kilobase, can be inserted into the double promoter vector. In one embodiment of the invention, the double promoter vector is derived from a VEE genomic RNA, and the second subgenomic promoter is a VEE subgenomic promoter. In an alternate embodiment, the double promoter vector is derived from a Sindbis (e.g., TR339) genomic RNA, and the second subgenomic promoter is a Sindbis (e.g., TR339) subgenomic promoter.

B. Replicon Vectors.

Replicon vectors, which are infectious, propagation-defective, virus vectors can also be used to carry out the present invention. Replicon vectors are described in more detail in WO 96/37616 to Johnston et al., U.S. Pat. No. 5,505,947 to Johnston et al., and U.S. Pat. No. 5,792,462 to Johnston et al; the disclosures of which are incorporated by reference herein in their entireties. Alphaviruses for constructing the replicon vectors according to the present invention include, but are not limited to, VEE, Semliki Forest Virus, S.A.AR86, Girdwood S.A., Sindbis (e.g., TR339), and Ockelbo.

In general, in the replicon system, one or more foreign gene(s) to be expressed is/are inserted in place of at least a portion of one or more of the viral structural protein genes in a transcription vector containing the viral sequences necessary for viral replication (e.g., the nsp1-4 genes). RNA transcribed from this vector contains sufficient viral sequences (e.g., the viral nonstructural genes) to be competent for RNA replication and transcription. This RNA can be transcribed in vitro or in vivo. In the case of in vitro transcribed RNA, the RNA is first transfected into susceptible cells by any method known in the art, wherein it is replicated and translated to give the replication proteins. These proteins will transcribe the transfected RNA, including the transgene(s), which will, optionally, be translated. In certain embodiments, the transgene(s) is/are operatively associated with the alphavirus 26S subgenomic promoter, which will produce high level of the transcript and, in the case of a translated RNA, the protein of interest. The autonomously replicating RNA (i.e., replicon) can only be packaged into virus particles if the deleted alphavirus structural protein genes are provided. The deleted alphavirus structural protein genes may be provided by any suitable means, e.g., by a stably transformed packaging cell line (see, e.g., U.S. Pat. No. 5,789,245), or by one or more helper nucleic acid molecules (RNA or DNA), which are provided to the cell along with the replicon vector, and are then expressed in the cell so that new replicon particles are produced in the cell.

In representative embodiments, the helper nucleic acids do not contain the viral nonstructural genes for replication, but these functions are provided in trans by the replicon molecule. In one embodiment, the non-structural proteins translated from the replicon molecule transcribe the structural protein genes on the helper nucleic acid molecule, resulting in the synthesis of viral structural proteins and packaging of the replicon into virus-like particles. As at least some of the alphavirus packaging or encapsidation signals are located within the nonstructural genes, the absence of these sequences in the helper nucleic acids precludes their incorporation into virus particles.

The replicon molecule is “propagation defective,” as described hereinabove inasmuch as the replicon RNA in these particles does not include all of the alphavirus structural proteins required for encapsidation, at least a portion of at least one of the required structural proteins being deleted therefrom. The replicon RNA therefore only initiates an abortive infection; no new viral particles are produced, and there is no spread of the infection to other cells.

Typically, the replicon molecule comprises an alphavirus packaging signal.

The replicon molecule is self-replicating. Accordingly, the replicon molecule comprises sufficient coding sequences for the alphavirus nonstructural polyprotein so as to support self-replication. In embodiments of the invention, the replicon encodes the alphavirus nsP1, nsP2, nsP3 and nsP4 proteins.

The replicon molecules of the invention “do not encode” one or more of the alphavirus structural proteins. By “do(es) not encode” one or more structural proteins, it is intended that the replicon molecule does not encode a functional form of one or more structural proteins and, thus, a complementing sequence is provided by a helper or packaging cell to produce new virus particles. In embodiments of the invention, the replicon molecule does not encode any of the alphavirus structural proteins.

The replicon may not encode the structural protein(s) because the coding sequence is partially or entirely deleted from the replicon molecule. Alternatively, the coding sequence is otherwise mutated so that the replicon does not express the functional protein. In embodiments of the invention, the replicon lacks all or substantially all of the coding sequence of the structural protein(s) that is not encoded by the replicon, e.g., so as to minimize recombination events with the helper sequences.

In particular embodiments, the replicon molecule may encode at least one, but not all, of the alphavirus structural proteins. For example, the alphavirus capsid protein may be encoded by the replicon molecule. Alternatively, one or both of the alphavirus glycoproteins may be encoded by the replicon molecule. As a further alternative, the replicon may encode the capsid protein and either the E1 or E2 glycoprotein.

In other particular embodiments, none of the alphavirus structural proteins are encoded by the replicon molecule. For example, all or essentially all of the sequences encoding the alphavirus capsid protein and glycoproteins may be deleted from the replicon molecule.

As yet another aspect, the invention provides a composition comprising a population of replicon particles containing no detectable replication-competent alphavirus particles. Replication-competent virus may be detected by any method known in the art, e.g., by neurovirulence following intracerebral injection into suckling mice, or by passage twice on alphavirus-permissive cells (e.g., BHK cells) and evaluation for virus induced cytopathic effects.

III. Attenuating Mutations.

The present invention also provides alphavirus virion coats (e.g., VEE virion coats) including attenuating mutations (as defined above) and genomic RNA and DNA constructs encoding the same. Those skilled in the art will appreciate that the alphaviruses of the invention may further comprise attenuating mutations in the nonstructural protein coding region or other regions of the alphavirus genome.

In particular embodiments, the attenuating mutation(s) reduces (e.g., by at least 25%, 40%, 50%, 60%, 70%, 80%, 90%, 95% or more) the neurovirulence of the alphavirus vector (e.g., as determined by intracerebral injection in weanling or adult mice). It is not necessary that the attenuating mutations of the invention eliminate all pathology or adverse effects associated with virus administration, as long as there is some improvement or benefit (e.g., increased safety and/or reduced morbidity and/or reduced mortality) as a result of the attenuating mutation.

Appropriate attenuating mutations will be dependent upon the alphavirus used. Exemplary attenuating mutations include, but are not limited to, those described in U.S. Pat. No. 5,505,947 to Johnston et al., U.S. Pat. No. 5,185,440 to Johnston et al., U.S. Pat. No. 5,643,576 to Davis et al., U.S. Pat. No. 5,792,462 to Johnston et al., and U.S. Pat. No. 5,639,650 to Johnston et al., the disclosures of which are incorporated herein in their entirety by reference.

Other attenuating mutations of particular interest include attenuating mutations in the E1 glycoprotein of the alphavirus virion shell (e.g., VEE virion shell). While not wishing to be bound by any theory of the invention, the E2 glycoprotein is believed to bind to cellular virus receptors and, thus, E1 mutants may advantageously achieve attenuation without disrupting cellular targeting. Accordingly, in embodiments of the invention, the attenuating mutation is a mutation in the E1 glycoprotein (e.g., the VEE E1 glycoprotein) that does not unduly interfere (e.g., reduce by more than 25%, 35% or 50%) with cellular targeting, receptor binding and/or infectivity, for example, to or in dendritic cells.

In other particular embodiments of the invention, the attenuating mutation is in the putative fusogenic peptide region in the alphavirus E1 glycoprotein (e.g., the fusogenic peptide region of the VEE E1 glycoprotein). This region is from about amino acid 80 to about amino acid 93 of the E1 glycoprotein and contains a stretch of uncharged and hydrophobic amino acids (see, e.g., Davis et al., (1994) Arch Virol [Suppl.] 9:99). Following virus binding via the E2 glycoprotein to the cell surface receptor, the glycoproteins rearrange and this hydrophobic domain is exposed and is believed to facilitate entry of the virus across the cellular membrane.

In particular embodiments, the alphavirus virion shell has an attenuating mutation at E1 glycoprotein amino acid position 81. For example, the attenuating mutation may be a phenylalanine to leucine or isoleucine mutation in Sindbis virus (e.g., strain TR339) or a mutation from tyrosine to leucine or isoleucine in Semliki Forest Virus or Ross River Virus. Similar mutations in the E1 fusogenic region may be made in any alphavirus (as defined above).

In other embodiments, the alphavirus comprises a VEE virion shell comprising an attenuating mutation at E1 glycoprotein amino acid position 81 and/or 253. The VEE virion shell may additionally contain other attenuating mutations. Attenuating mutations may be selected as described below. In particular embodiments, the attenuating mutation at amino acid position 81 is a mutation from phenylalanine to leucine or isoleucine. In other particular embodiments, the attenuating mutation at amino acid position 253 is a mutation from phenylalanine to serine or threonine.

Another particular attenuating mutation is an attenuating mutation in the VEE virion shell at E1 amino acid position 83. Typically, this attenuating mutation is used together with a second site suppressor mutation to avoid lethality.

One barrier encountered with many attenuating mutations is that the attenuated virus frequently has decreased immunogenicity, i.e., the virus is safer, but is less efficacious in eliciting the desired immune response. The present invention advantageously provides immunogenic compositions comprising attenuated alphavirus particles with improved efficacy (e.g., provides protection at a lower dosage) as compared with other attenuated alphaviruses. Methods of assessing the effectiveness of immunogenic compositions are well known in the art and include but are not limited to methods of evaluating protection against a challenge pathogen and indirect methods such as determination of antibody titers.

Thus, in particular embodiments, the present invention provides alphaviruses having attenuating mutations that achieve attenuation without significantly reducing (e.g., by more than 25%, 35% or 50%) immunogenicity, thereby resulting in a need for a corresponding increase in dosage. In particular embodiments, the present invention provides an attenuated alphavirus having a VEE shell, where the alphavirus is substantially as immunogenic as, or is even substantially more immunogenic than, a comparable alphavirus having a wild-type VEE virion shell (for example, the VEE 3000 described herein), i.e., a substantially similar number of infectious virus particles or even substantially less virus is required to provide an immunogenically effective dosage. By “substantially as immunogenic” it is intended that the attenuated alphavirus is as immunogenic as an alphavirus having a wild-type VEE coat (e.g., VEE 3000) at a dosage that is about 50% to 200% of the dosage of the virus having the wild-type VEE coat, i.e., one-half to two times as much attenuated virus is needed to elicit the same immune response as an alphavirus having a wild-type coat. In other embodiments, the alphavirus is “substantially more immunogenic” than a comparable alphavirus comprising a wild-type VEE coat, i.e., a substantially lower dosage (e.g., less than about 50%) of the attenuated virus provides the same immune response as the alphavirus comprising the wild-type VEE coat.

By “substantially less immunogenic” it is intended that the attenuated alphavirus is as immunogenic as an alphavirus having a wild-type VEE coat (e.g., VEE 3000) at a dosage that is about 250% or more of the dosage of a comparable alphavirus having a wild-type VEE coat, i.e., 2.5-times or more attenuated virus is needed to elicit the same immune response as an alphavirus having a wild-type coat. Because of the safety benefits of an attenuated virus, the concerns relating to administering high virus dosages to subjects, and the costs of virus production, alphaviruses having attenuated VEE coats that are less immunogenically effective than a comparable alphavirus having a wild-type VEE virion shell can nonetheless be advantageous and are encompassed by the present invention, e.g., attenuated viruses that require a dosage that is less than about 5ive-fold, less than about 7.5-fold, less than about 10-fold, less than about 15-fold, less than about 25-fold, less than about 50-fold higher, or even less than about 100-fold higher than the dosage of a comparable virus having a wild-type VEE virion shell to elicit a similar immune response.

Alternatively stated, in other embodiments of the invention, the attenuated virus is more immunogenic than a comparable attenuated virus comprising the 3014 VEE coat described below, i.e., a lower dosage of the attenuated virus of the invention produces an immunogenically effective response as compared with the dosage of an alphavirus comprising the 3014 coat. In particular embodiments, the immunogenically effective dosage of the attenuated virus of the invention is less than about 25%, about 50%, or about 75% of the dosage of a comparable virus having a 3014 VEE virion shell. In other embodiments, the immunogenically effective dosage of the attenuated virus is reduced by about one order of magnitude, two orders of magnitude, or even three orders of magnitude or more as compared with the dosage of a comparable virus having a 3014 VEE coat.

Those skilled in the art will appreciate that the relative immunogenicity of the attenuated alphavirus as compared with a suitable non-attenuated control virus (e.g., having a VEE 3000 coat) may vary depending upon the particular dosage, route of administration, species and age of the subject, and the like.

As described in Bernard et al., (2000) Virology 276:93, the wild type VEE virion shell only interacts poorly with heparin, whereas some attenuated VEE mutants (e.g., the 3014 mutant having an Ala→Thr mutation at E1 position 272, a Glu→Lys mutation at E2 position 209, and a Ile→Asn mutation at E2 position 239) bind relatively strongly to heparin. Methods of detecting viral interaction with heparin are known to those skilled in the art, for example, binding to immobilized heparin (e.g., a heparin column or beads) or inhibition of cell infectivity or binding by heparin (e.g., to BHK cells or dendritic cells), which are described in Bernard et al., (2000) Virology 276:93).

In some embodiments, the attenuated viruses of the invention do not exhibit detectable binding to, or only weakly bind to, heparin or heparan sulfate. According to these embodiments, the attenuated viruses of the invention are more similar to the wild-type virus than the 3014 mutant described above with respect to heparin binding. While not wishing to be bound by any particular theory, it appears that binding to heparin and/or heparan sulfate may increase viral clearance rates and reduce infectivity, with a resulting loss of immunogenicity. In other particular embodiments of the invention, the attenuated virus (e.g., an attenuated alphavirus with a VEE virion shell) does not exhibit detectable binding to glycosaminoglycans (e.g., heparin, heparan sulfate, chondroitin, chondroitin sulfate and/or dextran sulfate) or only exhibits weak binding thereto. Particular alphaviruses with attenuating mutations in the E2 glycoprotein and having only weak binding to heparin have been described by Bernard et al., (2000) Virology 276:93, the disclosures of which are incorporated by reference herein in its entirety.

In particular embodiments, the alphavirus comprises a VEE virion shell comprising an attenuating mutation in the E1 glycoprotein, where the alphavirus exhibits no detectable binding or only weak binding to heparin. In other embodiments, the alphavirus comprises a VEE virion shell comprising an attenuating mutation in the fusogenic peptide region of the E1 glycoprotein (as described above), wherein the alphavirus exhibits no detectable binding or only weak binding to heparin. The virion shell can further comprise additional attenuating mutations in the E2 and/or E3 glycoproteins (exemplary mutations in the E2 and E3 glycoproteins are discussed below).

In representative embodiments of the invention, the alphavirus comprises a VEE virion shell comprising an attenuating mutation at E1 amino acid position 81 and/or E1 253 (each as described above), and exhibits no detectable binding or only weak binding to heparin. For example, the 3042 mutation has a Phe→lie mutation at E1 position 81. Alternatively or additionally, the alphavirus comprises a VEE coat comprising an attenuating mutation that results in the deletion of the furin cleavage site in the E3 glycoprotein (e.g., deletion of E3 amino acids 56-59), and exhibits no detectable binding or only weak binding to heparin. This type of attenuating mutation may be present in conjunction with a second site mutation to maintain viability (e.g., a second site mutation at E1 amino acid position 253). Thus, in one particular embodiment, the attenuated mutant comprises a mutation (e.g., Phe→Ser) at E1 position 253 and a deletion of the furin cleavage site (e.g., deletion of E3 amino acids 56-59), and exhibits no detectable binding or only weak binding to heparin.

In still other embodiments, the attenuated alphavirus comprises a VEE virion shell comprising an attenuating mutation at E1 amino acid 272 (e.g., an Ala→Thr mutation). In further embodiments, the attenuated alphavirus comprises a VEE virion shell comprising attenuating mutations at E2 amino acids 76 and 166 (e.g., Glu→Lys mutation at E2 position 76 and a Lys→Glu mutation at E2 position 116).

As noted above, virus interaction with heparin may be assessed by inhibition of virus infectivity. In particular embodiments, a virus that “exhibits (only) weak binding” to heparin does not demonstrate a substantial reduction (i.e., more than about 50%) in infectivity (e.g., in BHK cells or dendritic cells) in the presence of relatively low concentrations of heparin (e.g., concentrations of about 50, 100, 150 or 200 μg/ml or less). In particular embodiments, heparin binds to the attenuated virus comprising the VEE virion shell (e.g., interfering with infectivity of the virus) with an affinity that is similar to or less than the affinity of heparin for the wild-type virus or, alternatively, is less than about two-fold, three-fold, four-fold, or five-fold greater than the affinity of the wild-type virion shell for heparin. Alternatively stated, in other embodiments, by “exhibits (only) weak binding” to heparin, it is meant that the affinity of heparin binding to the alphavirus comprising the attenuated VEE virion shell is less than about 25%, 20%, 15%, 10%, 5% or less than the affinity of the 3014 coat for heparin, e.g., interference of virus infectivity by heparin is less than about 25%, 20%, 15%, 10%, 5% or less than the interference of infectivity by a virus comprising the 3014 coat.

One of ordinary skill in the art may routinely identify attenuating mutations other than those specifically disclosed herein using methods known to those skilled in the art (see, e.g., Olmsted et al., (1984) Science 225:424 and Johnston and Smith (1988) Virology 162:437). Olmsted et al. describes a method of identifying attenuating mutations in Sindbis virus by selecting for rapid growth in cell culture. The Johnston and Smith publication describes the identification of attenuating mutations in VEE by applying direct selective pressure for accelerated penetration of BHK cells.

Likewise, one of ordinary skill in the art may routinely identify attenuating mutations having the desired characteristics (for example, improved immunogenicity as compared with known attenuating alphaviruses) using techniques for assessing immunogenicity known in the art (e.g., antibody titers may be measured by ELISA assay, hemagglutinin inhibition, virus neutralization and plaque reduction neutralization assays) and as described in the working examples herein.

The present invention also includes methods for identification of attenuating mutations that lack the ability to bind heparin and have increased immunogenicity. One such method involves the selection of virus particles with the ability to infect cell monolayers in vitro in the presence of heparin or heparan sulfate. In other embodiments of this method, other glycosaminoglycans can be used for this selection, including, but not limited to dextran sulfate, chondroitin sulfate A, chondroitin sulfate B as described in Klimstra et al. (1998) J. Virol. 72:7357-7366.

A spectrum of mutations are first engineered into the E1 and/or E2 glycoproteins of the alphavirus by methods well known in the art, such as random, site-directed or saturation mutagenesis. This heterogeneous population of mutated viral particles is then incubated with a permissive (i.e. a cell line that can be infected by the alphavirus) cell line in vitro in the presence of glycosaminoglycans at a sufficient concentration as to be inhibitory to the infection of the cell line by viral particles known to bind heparin, e.g., between 20 and 300 microgram/per ml. Alternatively, the viral population can be incubated with the glycosaminoglycan prior to exposure of the cell line to the mutant particles. This screening method selectively prohibits the entry of viral particles with significant affinity for the particular glycosaminoglycan and imposes selective pressure, allowing identification of low or non-binding glycosaminoglycan mutants that are able to enter the cell and establish a productive infection. These mutants are then passed for multiple passages through the cell line, under the same or increased stringencies of selection for non-glycosaminoglycan binding alphaviral shells. The selected mutant populations are isolated by plaque assay, plaque purified by methods known in the art to produce clonal populations of viral particles that are sequenced to identify individual and/or combinations of non-glycosaminoglycan binding mutations. These mutations, either separately or in combination, are introduced into the wild-type virus and further selected for their attenuation and potential increased immunogenicity by methods known in the art, e.g. Davis et al. 1991; U.S. Pat. No. 5,185,440; U.S. Pat. No. 5,505,947.

Another method for selecting attenuating mutations encompassed by this invention is to take the mutagenized viral population described above, which consists of a mixed population of alphaviral shell-mutated viruses, and select within this population using affinity-based chromatographic techniques, for example glycosaminoglycan matrix chromatographic columns (specifically heparin or any other glycosaminoglycan as described above). Low or non-glycosaminoglycan-binding mutant virus particles will pass through or elute from the column in the early fractions. Individual clonal viral populations are then isolated from these fractions by plaque purification. Purified viral clones are sequenced by standard methods to identify the specific mutations that can be introduced into the wild-type virus shell, and virus or replicon particles made with such mutated shells are assayed for both attenuation and immunogenicity. The overall stringency of this column selection method can be increased or decreased by methods known in the art such as altering column conditions, e.g. buffer pH, salt concentration, column length, and chromatographic matrix choice, to optimize the retention of glycosaminoglycan binding mutants and to expand the range of mutations that might be usefully employed in this invention.

Accordingly, the present invention encompasses other attenuating mutations that do not substantially reduce immunogenicity (i.e., the attenuated virus is essentially as immunogenic as, or more immunogenic than, a comparable alphavirus having a wild-type coat).

When the alphavirus structural proteins are from VEE, other suitable attenuating mutations may be selected from the group consisting of codons at E2 amino acid position 76 which specify an attenuating amino acid, preferably lysine, arginine, or histidine as E2 amino acid 76; codons at E2 amino acid position 120 which specify an attenuating amino acid, preferably lysine as E2 amino acid 120; codons at E2 amino acid position 209 which specify an attenuating amino acid, preferably lysine, arginine or histidine as E2 amino acid 209; codons at E1 amino acid 272 which specify an attenuating amino acid, preferably threonine or serine as E1 amino acid 272, as provided above.

Other suitable attenuated alphavirus vectors comprise an attenuating mutation in the capsid protease that reduces, preferably ablates, the autoprotease activity of the capsid and results, therefore, in non-viable virus. Capsid mutations that reduce or ablate the autoprotease activity of the alphavirus capsid are known in the art, see e.g., WO 96/37616 to Johnston et al., the disclosure of which is incorporated herein in its entirety. In particular embodiments, the alphavirus vector comprises a VEE capsid protein in which the capsid protease is ablated, e.g., by introducing an amino acid substitution at VEE capsid position 152, 174, or 226. Alternatively, one or more of the homologous positions in other alphaviruses may be altered to reduce capsid protease activity.

If the alphavirus vector comprises a Sindbis-group virus (e.g., Sindbis, S.A.AR86, GirdwoodSA, Ockelbo) capsid protein, the attenuating mutation may be a mutation at capsid amino acid position 215 (e.g., a Ser→Ala) that reduces capsid autoprotease activity (see, Hahn et al., (1990) J. Virology 64:3069).

In some embodiments, the alphavirus structural proteins are from S.A.AR86. Exemplary attenuating mutations in the S.A.AR86 structural proteins are known in the art (see, e.g., International Application No. PCT/US03/09121; incorporated by reference herein in its entirety).

To identify attenuating mutations other than those specifically disclosed herein, amino acid substitutions may be based on any characteristic known in the art, including the relative similarity or differences of the amino acid side-chain substituents, for example, their hydrophobicity, hydrophilicity, charge, size, and the like.

Amino acid substitutions other than those disclosed herein may be achieved by changing the codons of the genomic RNA sequence (or a DNA sequence), according to the following codon table:

TABLE 1

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC ACU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

In identifying other attenuating mutations, the hydropathic index of the amino acids may be considered. The importance of the hydropathic amino acid index in conferring interactive biologic function on a protein is generally understood in the art (see, Kyte and Doolittle, (1982) J. Mol. Biol. 157:105; incorporated herein by reference in its entirety). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, Id.), these are:

isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

Accordingly, the hydropathic index of the amino acid (or amino acid sequence) may be considered when identifying additional attenuating mutations according to the present invention.

It is also understood in the art that the substitution of amino acids can be made on the basis of hydrophilicity. U.S. Pat. No. 4,554,101 (incorporated herein by reference in its entirety) states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (±3.0); aspartate (+3.0±1); glutamate (+3.0±1); serine (+0.3); asparagine (+0.2); glutamine (+0.2); glycine (0); threonine (−0.4); proline (−0.5±l); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

Thus, the hydrophilicity of the amino acid (or amino acid sequence) may be considered when identifying additional attenuating mutations according to the present invention.

The attenuating mutations may be located in any of the structural proteins. The alphavirus vectors may contain two or more attenuating mutations within one structural protein or may contain two or more attenuating mutations distributed among the structural proteins. Further, additional attenuating mutations may be located on the replicon RNA in either the non-structural or structural coding regions as well as in non-coding regions.

Mutations may be introduced into the alphavirus vector by any method known in the art. For example, mutations may be introduced into the alphavirus RNA by performing site-directed mutagenesis on the cDNA which encodes the RNA, in accordance with known procedures (see, Kunkel, Proc. Natl. Acad. Sci. USA 82,488 (1985), the disclosure of which is incorporated herein by reference in its entirety). Alternatively, mutations may be introduced into the RNA by replacement of homologous restriction fragments in the cDNA which encodes for the RNA, in accordance with known procedures.

IV. Helper Cells, Helper Constructs and Methods of Producing Viral Particles.

Other aspects of the present invention are methods and helper cells for producing alphavirus particles in vitro. Methods and helper cells for producing alphavirus stocks, including double-promoter alphaviruses and alphavirus replicon particles are known in the art. See, e.g., U.S. Pat. No. 5,185,440 to Davis et al., U.S. Pat. No. 5,505,947 to Johnston et al.; U.S. Pat. No. 5,792,462 to Johnston et al., and Pushko et al. (1997) Virol. 239:389-401; the disclosures of which are incorporated herein by reference in their entireties. Methods for producing alphavirus particles using stably transformed packaging cell lines and/or DNA-based vector launches, such as the “ELVIS” system are also known in the art (see, e.g., U.S. Pat. No. 5,814,482 to Dubensky et al., U.S. Pat. No. 5,843,723 to Dubensky et al., U.S. Pat. No. 5,789,245 to Dubensky et al.; incorporated herein by reference in their entireties).

In representative embodiments, the methods and helper cells are used to produce propagation-incompetent alphavirus particles, for example, propagation-incompetent alphavirus replicon particles. According to this embodiment, the helper cells of the invention contain one or more helper nucleic acid sequences (e.g., as DNA and/or RNA molecules) encoding the alphavirus structural proteins (e.g., VEE structural proteins). The combined expression of the replicon molecule and the one or more helper molecules in the helper cell results in the production of an assembled alphavirus particle comprising a replicon RNA packaged within a virion comprising alphavirus structural proteins, which is able to infect a cell, but is unable to produce a productive infection (i.e., produce new virus particles).

In embodiments of the invention, the population of alphavirus particles produced according to the invention contains no detectable propagation-competent alphavirus particles. Propagation-competent virus may be detected by any method known in the art, e.g., by neurovirulence following intracerebral injection into suckling mice, or-by passage twice on alphavirus-permissive cells (e.g., BHK cells) and evaluation for virus induced cytopathic effects.

The helper cells are typically alphavirus-permissive cells. Alphavirus-permissive cells employed in the methods of the present invention are cells that, upon transfection with the viral RNA transcript, are capable of producing viral particles. Alphaviruses have a broad host range. Examples of suitable host cells include, but are not limited to fibroblasts, Vero cells, baby hamster kidney (BHK) cells, 293 cells, 293T cells, and chicken embryo fibroblast cells (e.g., DF-1 cells).

In particular embodiments, the helper cells of the invention may comprise sequences encoding the alphavirus structural proteins sufficient to produce an alphavirus particle, as described herein. Alternatively, or additionally, the helper cell may comprise a replicon RNA comprising one or more heterologous sequences, also as described herein.

As described hereinabove, in the production of a replicon particle, sequences encoding the alphavirus structural proteins are distributed among one or more helper molecules (preferably, two or three helper RNAs or DNAs). In addition, one or more structural proteins may be encoded by the replicon RNA, provided that the replicon RNA does not encode at least one structural protein such that the resulting alphavirus particle is propagation-incompetent in the absence of the helper sequence(s).

According to the present invention, at least one of the alphavirus structural and/or non-structural proteins encoded by the replicon and helper molecules contain one or more attenuating mutations, as described herein.

In one particular embodiment, the replicon molecule encodes at least one, but not all, of the alphavirus structural proteins (e.g., the E1 and/or E2 glycoproteins and/or the capsid protein). In one particular embodiment, the replicon encodes the capsid protein, and the E1 and E2 glycoproteins are encoded by one or more separate helper molecules. It may be advantageous to provide the glycoproteins by two separate helper molecules, so as to minimize the possibility of recombination to produce replication-competent virus.

In another embodiment, the replicon does not encode any of the E1 glycoprotein, the E2 glycoprotein, or the capsid protein. According to this embodiment, the capsid protein and alphavirus glycoproteins are encoded by one or more helper molecules, preferably two or more helper molecules. By distributing the coding sequences for the structural proteins among two, three or even more helper molecules, the likelihood that recombination will result in replication-competent virus is reduced.

In a further embodiment, the replicon does not encode any of the alphavirus structural proteins, and may lack the sequences encoding the alphavirus structural proteins.

As described above, the replicon may not encode the structural protein(s) because of a partial or complete deletion of the coding sequence(s) or otherwise contains a mutation that prevents the expression of a functional protein(s). In embodiments of the invention, all or substantially all of the coding sequences for the structural protein(s) that is not encoded by the replicon are deleted from the replicon molecule.

In one embodiment, the E1 and E2 glycoproteins are encoded by one helper molecule, and the capsid protein is encoded by another helper molecule. In another preferred embodiment, the E1 glycoprotein, E2 glycoprotein, and capsid protein are each encoded by separate helper molecules. In other embodiments, the capsid protein and one of the glycoproteins are encoded by one helper molecule, and the other glycoprotein is encoded by a second helper molecule.

In other particular embodiments, the helper and replicon sequences are RNA molecules that are introduced into the cell, e.g., by lipofection or electroporation. Uptake of helper RNA and replicon RNA molecules into packaging cells in vitro can be carried out according to any suitable means known to those skilled in the art. Uptake of RNA into the cells can be achieved, for example, by treating the cells with DEAE-dextran, treating the RNA with LIPOFECTIN™ before addition to the cells, or by electroporation, with electroporation being the currently preferred means. These techniques are well known in the art. See e.g., U.S. Pat. No. 5,185,440 to Davis et al., and PCT Publication No. WO 96/37616 to Johnston et al., the disclosures of which are incorporated herein by reference in their entirety.

Alternatively, one or all of the helper and/or replicon molecules are DNA molecules, which are either stably integrated into the genome of the helper cell or expressed from an episome (e.g., an EBV derived episome). The DNA molecule may be any vector known in the art, including but not limited to a non-integrating DNA vector, such as a plasmid, or a viral vector.

V. Recombinant Alphavirus Vectors.

According to embodiments of the invention, it is desirable to employ an alphavirus vector that encodes one or more (e.g., two, three, four, five, etc.) heterologous nucleic acid sequences, preferably each encoding an antigen according to the present invention. In particular embodiments, wherein there are two or more heterologous nucleotide sequences, each heterologous nucleic acid sequence will typically be operably associated with a promoter.

Alternatively, an internal ribosome entry site (IRES) sequence(s) can be placed downstream of the first heterologous nucleic acid sequence and upstream of a second or additional heterologous nucleic acid sequence(s). In any of these embodiments, the heterologous nucleic acid sequence(s) can be associated with a constitutive or inducible promoter. An exemplary promoter is an alphavirus 26S subgenomic promoter (e.g., VEE 26S subgenomic promoter). In general, the S.A.AR86 26S subgenomic promoter can be used with S.A.AR86 replication proteins, and the VEE 26S subgenomic promoter can be used with VEE replication proteins, and the like.

Heterologous nucleic acids of interest include nucleic acids encoding peptides and proteins, including immunogenic (e.g., for an immunogenic composition or a vaccine) or therapeutic (e.g., for medical or veterinary uses) polypeptides.

An “immunogenic” polypeptide, or “immunogen” as used herein is any polypeptide that elicits an immune response in a subject, and in particular embodiments, the immunogenic polypeptide is suitable for providing some degree of protection to a subject against a disease. The present invention may be employed to express an immunogenic polypeptide in a subject (e.g., for vaccination) or for immunotherapy (e.g., to treat a subject with cancer or tumors).

An immunogenic polypeptide, or immunogen, may be any polypeptide suitable for protecting the subject against a disease, including but not limited to microbial, bacterial, protozoal, parasitic, and viral diseases. For example, the immunogen may be an orthomyxovirus immunogen (e.g., an influenza virus immunogen, such as the influenza virus hemagglutinin (HA) surface protein or the influenza virus nucleoprotein gene, or an equine influenza virus immunogen), or a lentivirus immunogen (e.g., an equine infectious anemia virus immunogen, a Simian Immunodeficiency Virus (SIV) immunogen, or a Human Immunodeficiency Virus (HIV) immunogen, such as the HIV or SIV envelope GP160 protein, the HIV or SIV matrix/capsid proteins, and the HIV or SIV gag, pol, ref, tat, nef and env genes products). The immunogen may also be an arenavirus immunogen (e.g., Lassa fever virus immunogen, such as the Lassa fever virus nucleocapsid protein gene and the Lassa fever envelope glycoprotein gene), a Picornavirus immunogen (e.g., a Foot and Mouth Disease virus immunogen), a poxvirus immunogen (e.g., vaccinia, such as the vaccinia L1 or L8 genes), an Orbivirus immunogen (e.g., an African horse sickness virus immunogen), a flavivirus immunogen (e.g., a yellow fever virus immunogen, a West Nile virus immunogen, or a Japanese encephalitis virus immunogen), a filovirus immunogen (e.g., an Ebola virus immunogen, or a Marburg virus immunogen, such as NP and GP genes), a bunyavirus immunogen (e.g., RVFV, CCHF, and SFS immunogens), or a coronavirus immunogen (e.g., an infectious human coronavirus immunogen, such as the human coronavirus envelope glycoprotein gene, or a porcine transmissible gastroenteritis virus immunogen, or an avian infectious bronchitis virus immunogen). The immunogen may further be a polio antigen, tuberculosis antigen, herpes antigen (e.g., CMV, EBV, HSV antigens) mumps antigen, measles antigen, rubella antigen, diptheria toxin or other diptheria antigen, pertussis antigen, hepatitis (e.g., hepatitis A or hepatitis B) antigen, or any other vaccine antigen known in the art.

In embodiments of the invention, the antigen is Simian Immunodeficiency Virus (SIV) or Human Immunodeficiency Virus (HIV) antigen. For example, the antigen may be the product(s) of the SIV or HIV gag, env, ref, tat, nef or pol genes, or combinations thereof. In other particular embodiments, the antigen(s) is/are from a specific lade of the HIV virus, e.g., Clade B, C or E or combinations thereof.

Accordingly, in particular embodiments, the subject is a human subject or a simian subject that is infected with, or is at risk of becoming infected with HIV or SIV, respectively. Likewise, in other embodiments, the subject is a human subject that has, or is at risk of developing, AIDs.

The present invention may also be advantageously employed to produce an immune response against chronic or latent infective agents, which typically persist because they fail to elicit a strong immune response in the subject. Illustrative latent or chronic infective agents include, but are not limited to, hepatitis B, hepatitis C, Epstein-Barr Virus, herpes viruses, human immunodeficiency virus, and human papilloma viruses. Alphavirus vectors encoding antigens from these infectious agents may be administered to a cell or a subject according to the methods described herein.

Alternatively, the immunogen may be any tumor or cancer antigen. Preferably, the tumor or cancer antigen is expressed on the surface of the cancer cell. Exemplary cancer antigens for specific breast cancers are the HER2 and BRCA1 antigens. Other illustrative cancer and tumor cell antigens are described in S. A. Rosenberg; (1999) Immunity 10:281) and include, but are not limited to: MART-1/MelanA, gp100, tyrosinase, TRP-1, TRP-2, MAGE-1, MAGE-3, GAGE-1/2, BAGE, RAGE, NY-ESO-1, CDK-4, β-catenin, MUM-1, Caspase-8, KIAA0205, HPVE&, SART-1, PRAME, p15, and p53 antigens, and epitopes or fragments thereof. Additional cancer immunogens are the prostate-specific membrane antigen (PSMA), the prostate-specific antigen (PSA), CEA, or epitopes thereof.

The immunogen may also be a “universal” or “artificial” cancer or tumor antigen as described in international patent publication WO 99/51263, which is hereby incorporated by reference in its entirety.

The term “cancer” has its understood meaning in the art, for example, an uncontrolled growth of tissue that has the potential to spread to distant sites of the body (i.e., metastasize). Exemplary cancers include, but are not limited to, leukemias, lymphomas, colon cancer, renal cancer, liver cancer, breast cancer, lung cancer, prostate cancer, ovarian cancer, melanoma, and the like. Other illustrative cancers include cancers of the bone and bone marrow. Also encompassed are methods of treating and preventing tumor-forming cancers. The term “tumor” is also understood in the art, for example, as an abnormal mass of undifferentiated cells within a multicellular organism. Tumors can be malignant or benign. Preferably, the methods disclosed herein are used to prevent and treat malignant tumors.

Cancer and tumor antigens according to the present invention have been described hereinabove. Alphaviruses encoding cancer or tumor antigens may be administered in methods of treating cancer or tumors, respectively.

By the terms “treating cancer” or “treatment of cancer”, it is intended that the severity of the cancer is reduced or the cancer is at least partially eliminated. These terms may also indicate that metastasis of the cancer is reduced or at least partially eliminated. By the terms “prevention of cancer” or “preventing cancer” it is intended that the methods at least partially eliminate or reduce the incidence or onset of cancer. Alternatively stated, the present methods slow, control, decrease the likelihood or probability, or delay the onset of cancer in the subject.

Likewise, by the terms “treating tumors” or “treatment of tumors”, it is intended that the severity of the tumor is reduced or the tumor is at least partially eliminated. These terms may also indicate that metastasis of the tumor is reduced or at least partially eliminated. By the terms “prevention of tumors” or “preventing tumors” it is intended that the inventive methods at least partially eliminate or reduce the incidence or onset of tumors. Alternatively stated, the present methods slow, control, decrease the likelihood or probability, or delay the onset of tumors in the subject.

It is known in the art that immune responses may be enhanced by immunomodulatory cytokines (e.g., a-interferon, β-interferon, γ-interferon, ω-interferon, τ-interferon, interleukin-1α, interleukin-1β, interleukin-2, interleukin-3, interleukin-4, interleukin 5, interleukin-6, interleukin-7, interleukin-8, interleukin-9, interleukin-10, interleukin-11, interleukin 12, interleukin-13, interleukin-14, interleukin-18, B cell Growth factor, CD40 Ligand, tumor necrosis factor-α, tumor necrosis factor-β, monocyte chemoattractant protein-1, granulocyte-macrophage colony stimulating factor, and lymphotoxin). Accordingly, in particular embodiments of the invention, immunomodulatory cytokines (e.g., CTL inductive cytokines) are administered to a subject in conjunction with the methods described herein for producing an immune response or providing immunotherapy.

Cytokines may be administered by any method known in the art. Exogenous cytokines may be administered to the subject, or alternatively, a nucleotide sequence encoding a cytokine may be delivered to the subject using a suitable vector, and the cytokine produced in vivo. In preferred embodiments, an alphavirus vector encoding a cytokine is used to deliver the cytokine to the subject.

The present invention further finds use in methods of producing antibodies in vivo for passive immunization techniques. According to this embodiment, an alphavirus vector expressing an immunogen of interest is administered to a subject, as described herein by direct administration or ex vivo cell manipulation techniques. The antibody may then be collected from the subject using routine methods known in the art. The invention further finds use in methods of producing antibodies against an immunogen expressed from an alphavirus vector for any other purpose, e.g., for diagnostic purpose or for use in histological techniques.

The heterologous nucleic acid may be operably associated with expression control elements, such as transcription/translation control signals, origins of replication, polyadenylation signals, and internal ribosome entry sites (IRES), promoters, enhancers, and the like. Those skilled in the art will appreciate that a variety of promoter/enhancer elements may be used depending on the level and tissue-specific expression desired. The promoter/enhancer may be constitutive or inducible, depending on the pattern of expression desired. The promoter/enhancer may be native or foreign and can be a natural or a synthetic sequence.

Promoters/enhancers that are native to the subject to be treated are most preferred. Also preferred are promoters/enhancers that are native to the heterologous nucleic acid sequence. The promoter/enhancer is chosen so that it will function in the target cell(s) of interest. Mammalian promoters/enhancers are also preferred.

Preferably, the heterologous nucleotide sequence is operably associated with a promoter that provides high level expression of the heterologous nucleotide sequence, e.g., an alphavirus subgenomic 26S promoter (in particular, a VEE 26S subgenomic promoter).

In embodiments of the invention in which the heterologous nucleic acid sequence(s) will be transcribed and then translated in the target cells, specific initiation signals are generally required for efficient translation of inserted protein coding sequences. These exogenous translational control sequences, which may include the ATG initiation codon and adjacent sequences, can be of a variety of origins, both natural and synthetic.

VI. DNA Sequences, Vectors and Transformed Cells.

As a further aspect, the present invention provides DNA sequences (e.g., cDNA sequences) and vectors encoding infectious recombinant alphavirus genomic RNA transcripts (e.g., VEE genomic RNA transcripts) according to the present invention, comprising one or more heterologous nucleotide sequences. Also provided are alphavirus particles containing the recombinant alphavirus genomic RNA transcribed from the DNA molecules.

The present invention further provides vectors comprising a DNA sequence encoding a recombinant alphavirus genomic RNA transcript operably associated with a promoter that drives transcription of the DNA sequence. Examples of promoters which are suitable for use with the DNA sequences of the present invention include, but are not limited to T3 promoters, T7 promoters, cytomegalovirus (CMV) promoters, and SP6 promoters.

The DNA sequence may be encoded by any suitable vector known in the art, including but not limited to, plasmids, naked DNA vectors, yeast artificial chromosomes (yacs), bacterial artificial chromosomes (bacs), phage, viral vectors, and the like.

Genomic RNA transcripts may be synthesized from the DNA template by any method known in the art. For example, the RNA can be synthesized from the DNA sequence in vitro using purified RNA polymerase in the presence of ribonucleotide triphosphates and cap analogs in accordance with conventional techniques. Alternatively, the RNA may be synthesized intracellularly after introduction of the DNA.

Further provided are cells containing the DNA sequences, genomic RNA transcribed from the DNA sequences, and alphavirus vectors of the invention. Exemplary cells include, but are not limited to, fibroblast cells, Vero cells, Baby Hamster Kidney (BHK) cells, Chinese Hamster Ovary (CHO) cells, 293 cells, 293T cells, and chicken embryo fibroblast cells (e.g., DF-1 cells), macrophages, PBMC, monocytes, and dendritic cells.

The alphavirus DNA constructs, genomic RNA transcripts, and virus particles produced therefrom are useful for the preparation of pharmaceutical formulations, such as vaccines. In addition, the DNA clones, genomic RNA transcripts, and infectious viral particles of the present invention are useful for administration to animals for the purpose of producing antibodies to the alphavirus, which antibodies may be collected and used in known diagnostic techniques for the detection of alphaviruses. Antibodies can also be generated to the viral proteins expressed from the DNAs disclosed herein. As another aspect of the present invention, the claimed DNA clones are useful as nucleotide probes to detect the presence of alphavirus transcripts.

VII. Subjects, Pharmaceutical Formulations, Vaccines, and Modes of Administration.

The present invention finds use in both veterinary and medical applications. Suitable subjects include both avians and mammals, with mammals being preferred. The term “avian” as used herein includes, but is not limited to, chickens, ducks, geese, quail, turkeys and pheasants. The term “mammal” as used herein includes, but is not limited to, primates (e.g., simians and humans), bovines, ovines, caprines, porcines, equines, felines, canines, lagomorphs, rodents (e.g., rats and mice), etc. Human subjects include fetal, neonatal, infant, juvenile and adult subjects.

The invention may be used in either a therapeutic or prophylactic manner. For example, in one embodiment, to protect against an infectious disease, subjects may be vaccinated prior to exposure, as neonates or adolescents. Adults that have not previously been exposed to the disease may also be vaccinated. In cancer patients, use of the present invention may be used in conjunction with other cancer therapies, e.g., before, during or after the surgical removal of tumors, chemotherapy or radiation.

In particular embodiments, the present invention provides a pharmaceutical composition comprising an alphavirus vector of the invention in a pharmaceutically-acceptable carrier or other medicinal agents, pharmaceutical agents, carriers, adjuvants, diluents, etc. For injection, the carrier will typically be a liquid. For other methods of administration, the carrier may be either solid or liquid, such as sterile, pyrogen-free water or sterile pyrogen-free phosphate-buffered saline solution. For inhalation administration, the carrier will be respirable, and will preferably be in solid or liquid particulate form. As an injection medium, it is preferred to use water that contains the additives usual for injection solutions, such as stabilizing agents, salts or saline, and/or buffers.

In other embodiments, the present invention provides a pharmaceutical composition comprising a cell (e.g., a dendritic cell) that has been infected and genetically modified by an alphavirus vector in a pharmaceutically-acceptable carrier or other medicinal agents, pharmaceutical agents, carriers, adjuvants, diluents, etc.

By “pharmaceutically acceptable” it is meant a material that is not biologically or otherwise undesirable, e.g., the material may be administered to a subject without causing any undesirable biological effects. Thus, such a pharmaceutical composition may be used, for example, in transfection of a cell ex vivo or in administering the alphavirus/antibody compositions or cells directly to a subject.

The cell to be administered the virus vectors can be of any type, including but not limited to neuronal cells (including cells of the peripheral and central nervous systems), retinal cells, epithelial cells (including dermal, gut, respiratory, bladder and breast tissue epithelium), muscle cells (including cardiac, smooth muscle, skeletal muscle, and diaphragm muscle), pancreatic cells (including islet cells), hepatic cells (e.g., parenchyma), fibroblasts, endothelial cells, germ cells, lung cells (including bronchial cells and alveolar cells), prostate cells, stem cells, progenitor cells, dendritic cells, and the like. Alternatively, the cell is a cancer cell (including tumor cells). Moreover, the cells can be from any species of origin, as indicated above.

Alternatively, in embodiments of the invention, the cell is preferably a cell is a bone marrow cell or a cell in the bone-associated connective tissue. Other preferred cells, are cells of the periosteum, endosteum and tendons, generally within the epiphyses of the long bones adjacent to joints.

In still other embodiments, the cell is an antigen-presenting cell (e.g., a dendritic cell or a macrophage).

Cells that may be infected by the alphavirus vectors of the present invention further include, but are not limited to, polymorphonuclear cells, hemopoietic stem cells (including megakaryocyte colony forming units (CFU-M), spleen colony forming units (CFU-S), erythroid colony forming units (CFU-E), erythroid burst forming units (BFU-E), and colony forming units in culture (CFU-C), erythrocytes, macrophages (including reticular cells), monocytes, granulocytes, megakaryoctyes, lymphocytes, fibroblasts, osteoprogenitor cells, osteoblasts, osteoclasts, marrow stromal cells, chondrocytes and other cells of synovial joints.

The alphavirus vectors of the invention may be administered to elicit an immunogenic response (e.g., as an immunogenic composition or as a vaccine for immunotherapy). Typically, immunological compositions of the present invention comprise an immunogenic amount of infectious virus particles as disclosed herein in combination with a pharmaceutically-acceptable carrier.

An “immunogenic amount” is an amount of the infectious virus particles that is sufficient to induce an immune response in the subject to which the pharmaceutical formulation is administered. Typically, a dosage of about 10³ to about 10¹⁵ infectious units, about 10⁴ to about 10¹⁰ infectious units, about 10² to about 10⁶ infectious units; about 10³ to about 10⁵ infectious units, about 10⁵ to about 10⁹ infectious units, or about 10⁶ to about 10⁸ infectious units per dose is suitable, depending upon the age and species of the subject being treated, and the immunogen against which the immune response is desired.

In other embodiments, a dosage of about 10³ to about 10⁴ infectious units, about 10⁴ to about 10⁵ infectious units, about 10⁴ to about 10⁶ infectious units, about 10⁶ to about 10⁷ infectious units, about 10⁷ to about 10⁸ infectious units, about 10⁶ to about 10⁷ infectious units, about 10⁹ to about 10¹⁰ infectious units, or about 10¹⁰ to about 10¹¹ infectious units per dose is suitable.

In still other embodiments, the dosage is about 10³ to about 5×10³ infectious units, about 5×10³ to about 10⁴ infectious units, about 10⁴ to about 5×10⁴ infectious units, about 5×10⁴ to about 10⁵ infectious units, about 10⁵ to about 5×10⁵ infectious units, about 5×10⁵ to about 10⁶ infectious units, about 10⁶ to about 5×10⁶ infectious units, about 5×10⁶ to about 10⁷ infectious units, about 10⁷ to about 5×10⁷ infectious units, about 10⁷ to about 5×10⁷ infectious units, about 5×10⁷ to about 10⁸ infectious units, about 10⁸ to about 5×10⁸ infectious units, or about 5×10⁸ to about 10⁹ infectious units per dose.

In yet further embodiments, the dosage is about 10³, about 10⁴, about 10⁵, about 10⁶, about 10⁷, about 10⁸, about 10⁹, or about 10¹⁰ infectious units per dose.

Subjects and immunogens are as described above. In representative embodiments, the alphavirus vector is an alphavirus replicon particle (e.g., a VEE replicon particle).

The terms “vaccination” or “immunization” are well-understood in the art. For example, the terms vaccination or immunization can be understood to be a process that increases a subject's immune reaction to antigen and therefore the ability to resist or overcome infection. In the case of the present invention, vaccination or immunization may also increase the organism's immune response and resistance to invasion by cancer or tumor cells.

Any suitable vaccine and method of producing an immune response (i.e., immunization) known in the art may be employed in carrying out the present invention, as long as an active immune response (preferably, a protective immune response) against the antigen is elicited.

According to the present invention, administration of an alphavirus vector comprising one or more heterologous nucleotide sequences encoding an immunogen elicits an active immune response in the subject, and in particular embodiments, the active immune response is a protective immune response.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to immunogens by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

A “protective” immune response or “protective” immunity as used herein indicates that the immune response confers some benefit to the subject in that it prevents or reduces the incidence of disease. Alternatively, a protective immune response or protective immunity may be useful in the treatment of disease, in particular cancer or tumors (e.g., by causing regression of a cancer or tumor and/or by preventing metastasis and/or by preventing growth of metastatic nodules). The protective effects may be complete or partial, as long as the benefits of the treatment outweigh any disadvantages thereof.

Vaccination can be by any means known in the art, but is preferably by oral, rectal, transmucosal, intranasal, topical, transdermal, inhalation, parenteral (e.g., intravenous, subcutaneous, intradermal, intramuscular, intraperitoneal and intraarticular) administration, and the like. Alternatively, the alphavirus vector may be directly administered by implant or injection into or near a tumor. In the case of animal subject, injection may be into the footpad.

In particular embodiments of the invention, administration is by subcutaneous or intradermal administration. Subcutaneous and intradermal administration may be by any method known in the art, including but not limited to injection, gene gun, powderject device, bioject device, microenhancer array, microneedles, and scarification (i.e., abrading the surface and then applying a solution comprising the virus).

In other embodiments, administration is to the limb of the subject, e.g., by subcutaneous or intradermal administration. In still other particular embodiments, administration to the limb (e.g., by subcutaneous or intradermal routes) is to the front limb of the subject, i.e., in the case of bipeds such as a primate, administration is to the arm of the subject and in the case of a quadruped, administration is to the front leg. In still further embodiments, administration is to the lower part of the arm (e.g., in a primate, below the elbow).

Injectables can be prepared in conventional forms, either as liquid solutions or suspensions, solid forms suitable for solution or suspension in liquid prior to injection, or as emulsions. Alternatively, one may administer these reagents as an aerosol, or in a local rather than systemic manner, for example, in a depot or sustained-release formulation.

In other preferred embodiments, the alphavirus vector is administered intramuscularly, more preferably by intramuscular injection or by local administration (as defined above).

In other preferred embodiments, the alphavirus vectors of the present invention are administered to the lungs. The alphavirus vectors disclosed herein may be administered to the lungs of a subject by any suitable means, but are preferably administered by administering an aerosol suspension of respirable particles comprised of the alphavirus vectors, which the subject inhales. The respirable particles may be liquid or solid. Aerosols of liquid particles comprising the alphavirus vectors may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the virus vectors may likewise be produced with any solid particulate medicament aerosol generator, by techniques known in the pharmaceutical art.

The present invention further provides a method of delivering a nucleic acid to a cell (e.g., to produce an immune response or for therapy). For in vitro methods, the virus may be administered to the cell by standard viral transduction methods, as are known in the art. Cells to be administered the alphavirus vector are as described above. Preferably, the virus particles are added to the cells at the appropriate multiplicity of infection according to standard transduction methods appropriate for the particular target cells. Titers of virus to administer can vary, depending upon the target cell type and the particular virus vector, and may be determined by those of skill in the art without undue experimentation.

In particular embodiments of the invention, cells are removed from a subject, the alphavirus vector is introduced therein, and the cells are then replaced back into the subject. Methods of removing cells from subject for treatment ex vivo, followed by introduction back (e.g., intravenously) into the subject are known in the art. Alternatively, the alphavirus vector is introduced into cells from another subject, into cultured cells, or into cells from any other suitable source, and the cells are administered to a subject in need thereof. Preferably, if the subject's own cells are not used, the cells are HLA compatible with the subject's HLA type. The modified cell may be administered according to a method of ex vivo gene therapy or to provide immunity to a subject (e.g., by introducing a nucleotide sequence encoding an immunogen into an antigen producing cell, such as a dendritic cell).

Dosages of the cells to administer to a subject will vary upon the age, condition and species of the subject, the type of cell, the nucleic acid being expressed by the cell, the mode of administration, and the like. Typically, at least about 10² to about 10⁸, preferably about 10³ to about 10⁶ cells, will be administered per dose. Preferably, the cells will be administered in a “immunogenic amount” (as described hereinabove) or a “therapeutically-effective amount”.

Particular embodiments of the present invention are described in greater detail in the following non-limiting examples.

### EXAMPLE 1

### Materials and Methods

Virus: VEE replicon particles (VRP) expressing either influenza virus hemagglutinin (HA-VRP-3000, HA-VRP-3014, and HA-VRP-3042), green fluorescent protein (GFP-VRP-3000, GFP-VRP-3014, and GFP-VRP-3042), or HIV Clade C gag (HIV_{gag}-VRP-3000, HIV_{gag}-VRP-V3014, and HIV_{gag}-VRP-3042) were prepared as previously described (MacDonald and Johnston, 2000 J. Virology 74:914, Pushko et al. 1997 Virology 239:389). Briefly, RNA transcripts from replicon cDNA plasmids encoding the appropriate heterologous gene were co-electroporated with RNA transcripts from two helper constructs encoding either VEE capsid or VEE glycoprotein genes into baby hamster kidney (BHK) cells. VRP were harvested directly from the culture supernates 24 hr following electroporation and titered on BHK cells. For these studies, VRP were produced using a glycoprotein helper that contained the V3014 attenuating mutations, i.e., an Ala→Thr mutation at E1 position 272, a Glu→Lys mutation at E2 position 209, and a Ile→Asn mutation at E2 position 239 (Davis et al., (1991) Virology 183:20), V3040 attenuating mutation at E1 253 (Phe→Ser) or the V3042 attenuating mutation at E1 81 (Phe→Ile).

Mice and Cells: Seven- to eight-week-old female CD1 out bred mice (Charles River Laboratory) were inoculated subcutaneously (sc) in the left rear foot pad with 5×10⁵ infectious units (IU) of VEE viral replicon particles (VRP) unless otherwise specified. Mice were perfused with 4% paraformaldehyde (PFA) in PBS 24 hr post-inoculation (pi) and the draining popliteal lymph nodes were removed to PFA. Fixed frozen sections were analyzed by fluorescent microscopy for cells expressing GFP.

Bone marrow (BM) cells were isolated from the femurs of C57BL6 mice. Cells were grown as previously described. Briefly, marrow was flushed from femurs and tibia and resuspended in PBS. Cells were washed and re-suspended in RPMI1640 supplemented with 10% FBS, L-glutamine, nonessential amino acids, sodium pyruvate, 50 μM β-2-mercaptoethanol, and 25 mM HEPES. Cultures were supplemented with 0.1 ng/ml GM-CSF alone or with either 5% conditioned culture medium from the epidermal fibroblast cell line, NS46 (Xu et al., (1995) J. Immunol. 154:2697) or 1 ng/ml IL4 and grown on standard tissue culture plates.

VEE Replicon Particles (VRP) Inoculation of Macaques: VEE replicon particles packaged using wild-type glycoprotein coats were inoculated into rhesus macaques in each leg (5 cm lateral to the inguinal triangle) with 1×10⁴ or 1×10⁷ IU VRP-GFP or VRP-HA in 0.5 ml PBS. Inguinal lymph nodes were harvested 18 hours post inoculation, fixed immediately in paraformaldehyde, and processed for microscopy.

ELISA: Antibody assays were performed as described in Davis et al. (1996) J. Virol. 70:3781-3787. Gradient-purified PR/8/34 influenza virus was used as an antigen and horseradish peroxidase (HRP)-conjugated anti-mouse immunoglobulin G (IgG) or HRP-conjugated goat anti-mouse IgA was used as the second antibody.

In Situ Hybridization Analysis: Tissues were prepared as described by Charles et al. (1995) Virology 208:662-671 and Grieder et al. (1995) Virology 206:994-1006. Assays were performed as described in Davis et al. (1996) J. Virol. 70:3781-3787.

### EXAMPLE 2

### Anti-HA Response to VRP Immunization

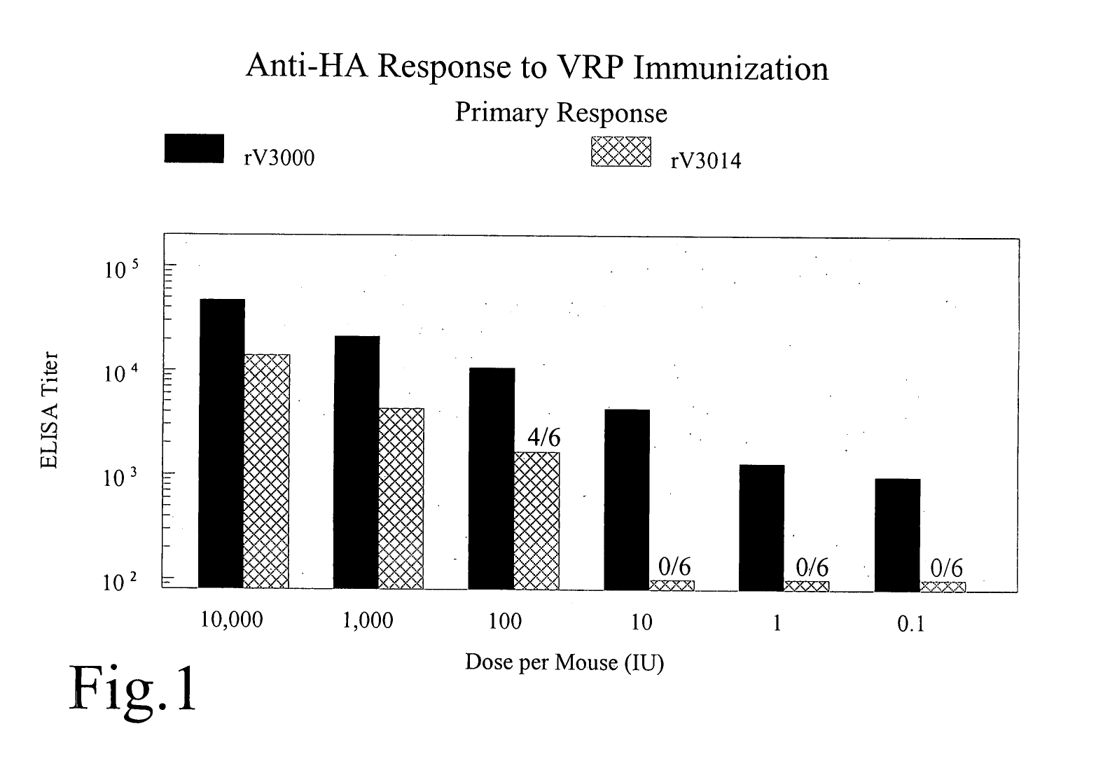

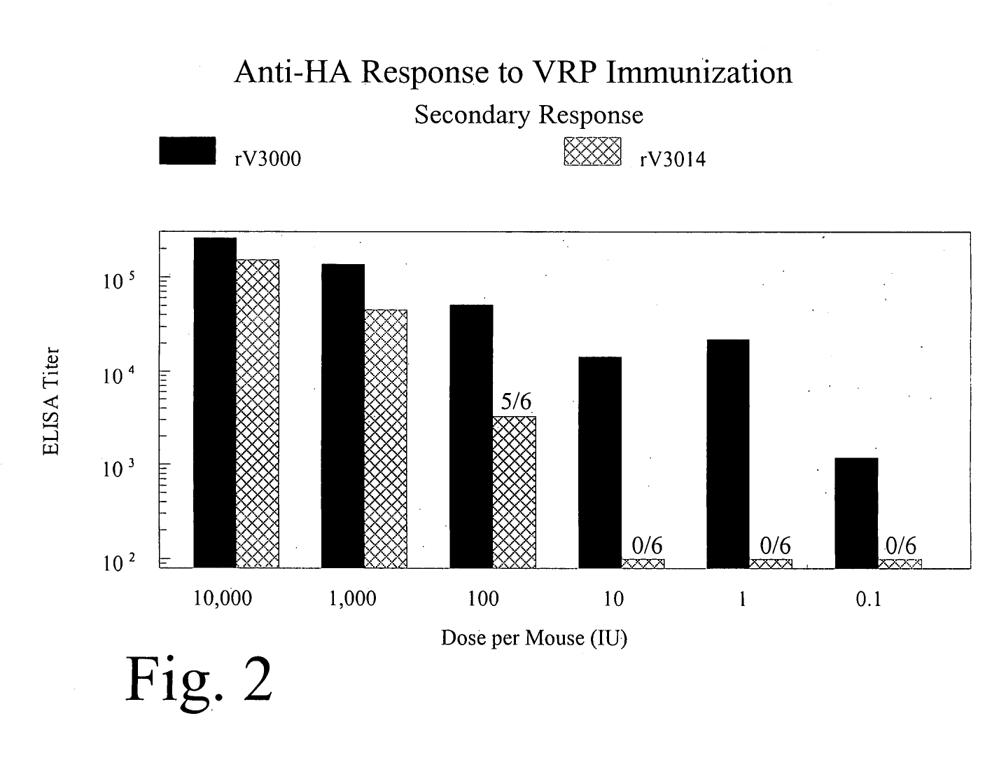

The effect of dosage on the primary and secondary response in HA vector-immunized mice was examined. VRP-replicons were administered at 0.1 to 10,000 IU. Four weeks post-inoculation, the mice were bled and ELISA assays for anti-HA response at varying doses of HA-VRP-3000 (wild-type) and HA-VRP-3014 (attenuated) were performed. The results are depicted in FIG. 1. In the same animals at four weeks, a second inoculation of VRP was administered. Four weeks after the second inoculation, ELISA assays for secondary Anti-HA response were performed and are shown in FIG. 2. These results indicate that mutations in the coat protein have a significant effect on the HA replicon induced immune response. At or below a dose of 10 IU per mouse, little primary or secondary response from immunization with HA-VRP-3014 (mutant coat protein) was observed in comparison to HA-VRP-3000 (wild-type). As the vector dosage is increased (100-10,000 IU), response to HA-VRP-3014 as determined from ELISA titer improves in both primary and secondary responses. The secondary response to HA-VRP-3014 at a dose of 10,000 IU approached that of the wild-type (HA-VRP-3000).

### EXAMPLE 3

### HIV Clade C Gag-Specific CTL Response in Mice

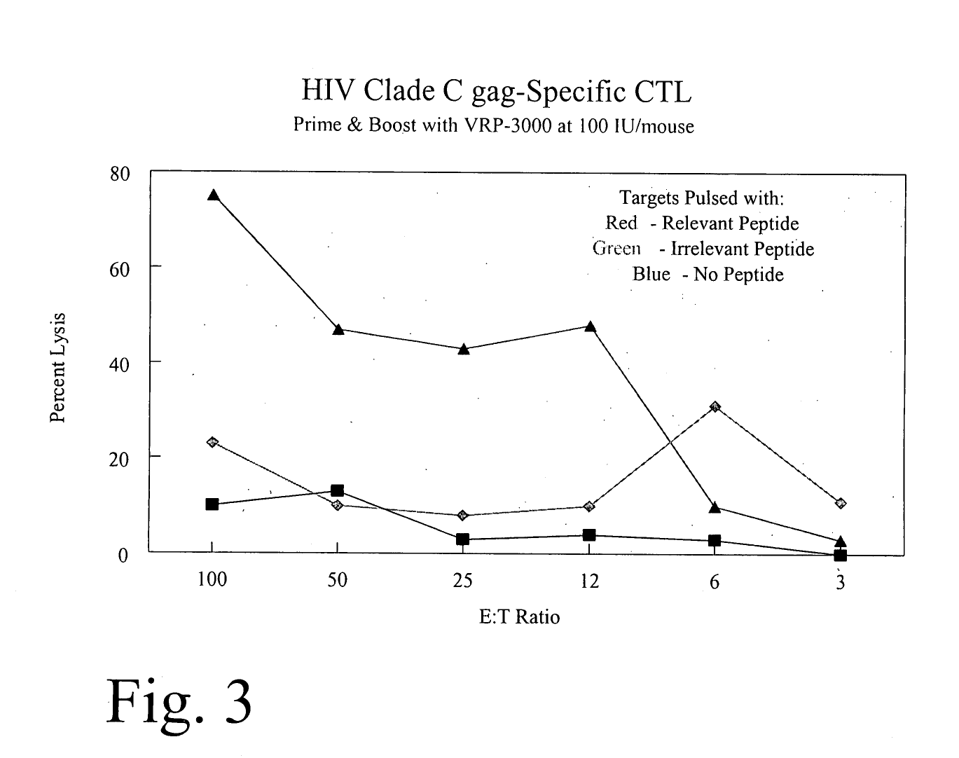

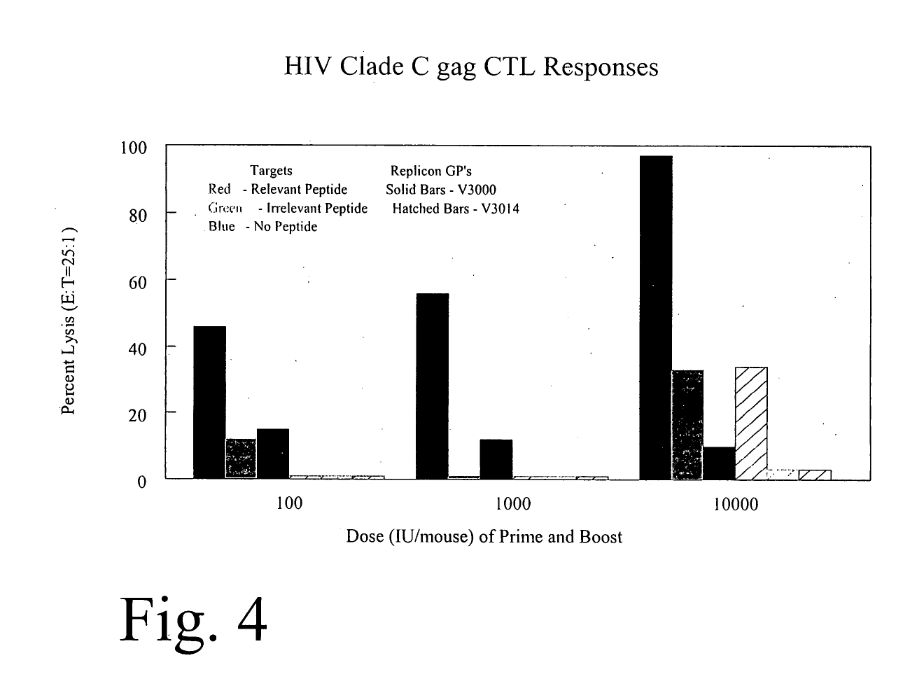

CTL response to HIV Clade gag in mice primed and boosted with 100 IU of HIV_{gag}-VRP-3000 is depicted in FIG. 3. Groups of six mice were primed and boosted four weeks after initial inoculation. HIV_{gag}-specific CTL responses were determined according to a standard chromium release assay (Hioe and Frelinger (1995) Mol. Immunol. 32:725-731) one week following the boost at various effector to target (E:T) cell ratios. A Class 1 H-2 K^{d} restricted Gag peptide (AMQMLKETI) was used as the relevant peptide. An irrelevant H-2K^{d} restricted HA (influenza virus hemagglutinin) peptide was used as a negative control. The percent specific lysis was calculated as:
[(experimental release−spontaneous release)/(maximum release−spontaneous release)]×100.
Spontaneous release was defined as counts per minute released from target cells in the absence of effector cells, and maximum release was defined as counts per minute released from target cells lysed with 2.5% Triton X-100. HIV_{gag}-specific CTL activity was defined as 10% lysis above controls. The results shown in FIG. 3 indicate that HIV_{gag}-VRP replicons can induce a HIV_{gag}-specific CTL response. The CTL response to HIV Clade gag in mice primed and boosted with HIV_{gag}-VRP replicons packaged in different coat proteins (wild-type HIV_{gag}-VRP-3000 and mutant HIV_{gag}-VRP-3014) at varying doses is depicted in FIG. 4. These results indicate that the replicon coat protein has an effect on the observed CTL response in primed and boosted mice. VRP-3014 (mutant coat proteins) elicits a weaker CTL response than VRP-3000 (wild-type).

### EXAMPLE 4

### Envelope Effect on HA Response in Mice

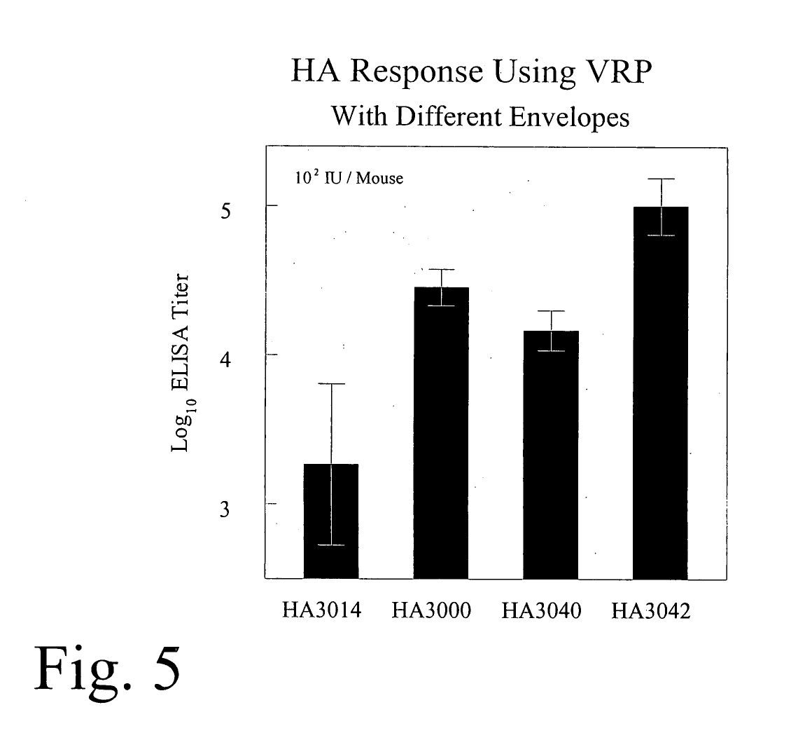

The effect of envelope coat protein on HA replicon induced immunogenicity is shown in FIG. 5. ELISA titers comparing HA response to HA replicons with different envelopes indicate that mutations in the coat protein do not necessarily have deleterious effects on antibody response. The E1 81 mutation HA3042 elicits a greater HA response than even the wild-type HA3000, while HA3014 elicits weaker responses than the wild-type. HA3040 exhibits only a modest depression as compared with the wild-type. These results suggest that the attenuated coat viruses, 3040 and 3042, are safe without substantially adverse effects on efficacy.

### EXAMPLE 5

### Effect of Route of Administration on HA Response in Mice

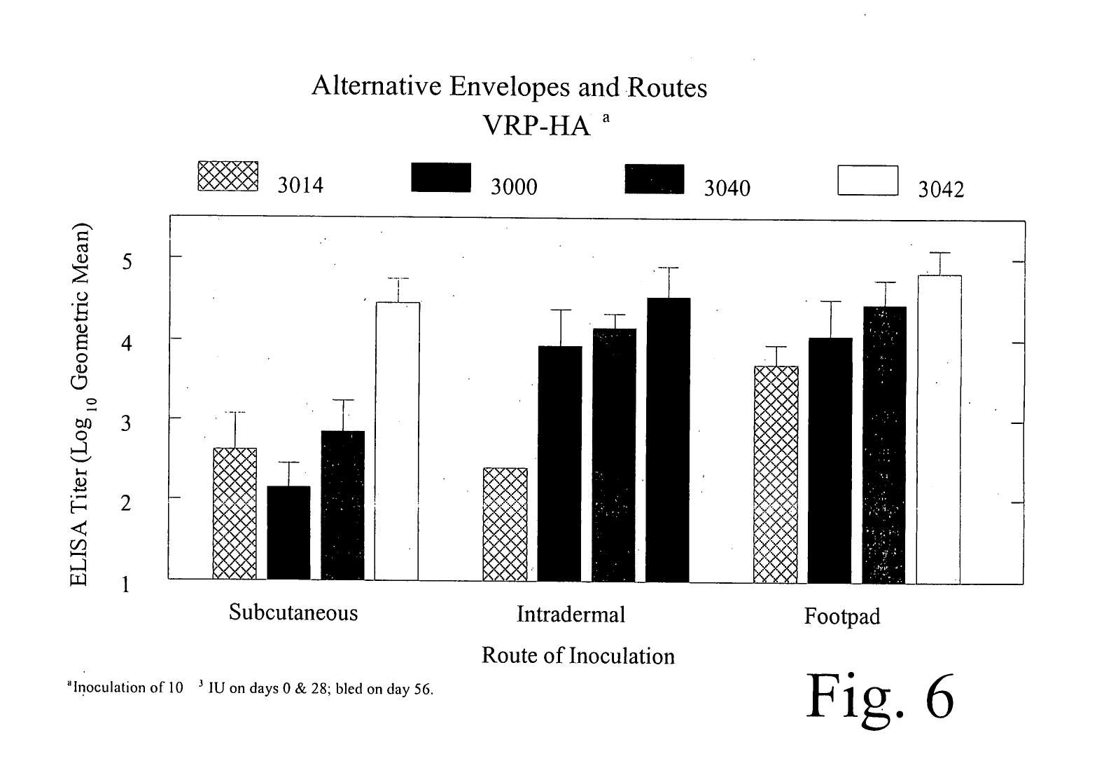

HA replicons were introduced by subcutaneous inoculation in the back of the neck, and by intradermal inoculation in the rear thigh. Four weeks following the first inoculation with 10³ IU VRP, a second 10³ IU dose of VRP was administered, and the mice were bled four weeks thereafter. The ELISA antibody titers are shown in FIG. 6. The results indicate that intradermal inoculation of HA-VRP generally elicits a stronger secondary response than subcutaneous inoculation. HA3042 produced a strong response by all routes of administration. In contrast, wild type, HA3014 and HA3040 gave a stronger response with intradermal administration as compared with subcutaneous administration. Wild type and attenuated viruses elicited a strong response with inoculation via the footpad. In all cases, HA3042 elicits the strongest ELISA response. The difference in response is most apparent in subcutaneous inoculations, with lesser differences observed for intradermal and footpad inoculations.

### EXAMPLE 6

### Dosage and Route Effect on Dendritic Cell Infection in Macaques

GFP-VRP-3000 is administered to four rhesus macaques by either subcutaneous or intradermal inoculation, 5 cm lateral to the inguinal triangle. Two animals receive a high dose (10⁷ IU of VRP), and two animals receive a low dose (10⁴ IU of VRP) of vector. The right leg of each animal receives a subcutaneous inoculation of vector, while the left leg receives an intradermal inoculation of vector. Eighteen hours post-inoculation, simple excision of the inguinal lymph nodes is performed and processed for fluorescence microscopy. The results from the fluorescence microscopy performed on these tissues indicates the effect of the route (subcutaneous vs. intradermal) and dosage on dendritic cell infection.

### EXAMPLE 7

### Quantitation of Immune Response to Vaccination in Macaques

HA-VRP-3000 is administered at 10⁵ IU in 0.5 ml PBS to two groups of four animals and boosted at 1 month. One group of animals receives the vaccine via subcutaneous inoculation, the other group receives the vaccine via intradermal inoculation. Inoculations are performed as outlined in Example 6. Blood is drawn for antibody determinations (anti-HA) at 0, 1, 2, and 4 months by ELISA. The results from this study allow the direct quantification of the immune response resulting from the different routes of vaccine administration.

### EXAMPLE 8

### Effect of Coat Protein on Dendritic Cell Infection in Macaques

GFP-VRP-3000 (wild-type coat protein), along with GFP-VRP-3014 and GFP-VRP-3042 (mutant coat proteins) are used in this study. The study is divided into two groups: high dose (10⁷ IU), and low dose (10⁴ IU). Each animal receives one dose of vaccine (in 0.5 ml PBS) in each leg (5 cm lateral from the inguinal triangle) via the most effective route of administration as determined in Example 6. Each animal (twelve total) are vaccinated in the following scheme:

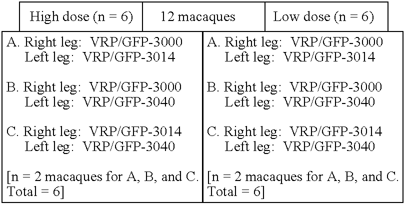

Simple excision of inguinal lymph nodes is performed from both sides using sterile technique and standard surgical methods 18 hours post-inoculation. The nodes are immediately be fixed in paraformaldehyde and processed for microscopy. The results examine the effect of dose and VRP coat protein on dendritic cell targeting of VRP infection.

### EXAMPLE 9

### Effect of Coat Protein on Immune Response in Macaques

HA-VRP-3000 (wild-type coat protein), along with HA-VRP-3014 and HA-VRP-3042 (mutant coat proteins) are used in this study. Three groups of four animals are used in this study, the first group is inoculated with HA-VRP-3000, the second group is inoculated with HA-VRP-3014, and the third group is inoculated with HA-VRP-3042. Each animal is inoculated with 10⁵ IU in 0.5 ml PBS of the appropriate vector at 0 and 1 month via the most effective route as determined according to Example 6. Animals are bled at 0, 1, 2, and 4 months for Anti-HA response. The results correlate the effect of VRP coat protein on immune response elicited by the vaccine.

### EXAMPLE 10

### Dendritic Cell Infection in Draining Lymph Nodes of Macaques

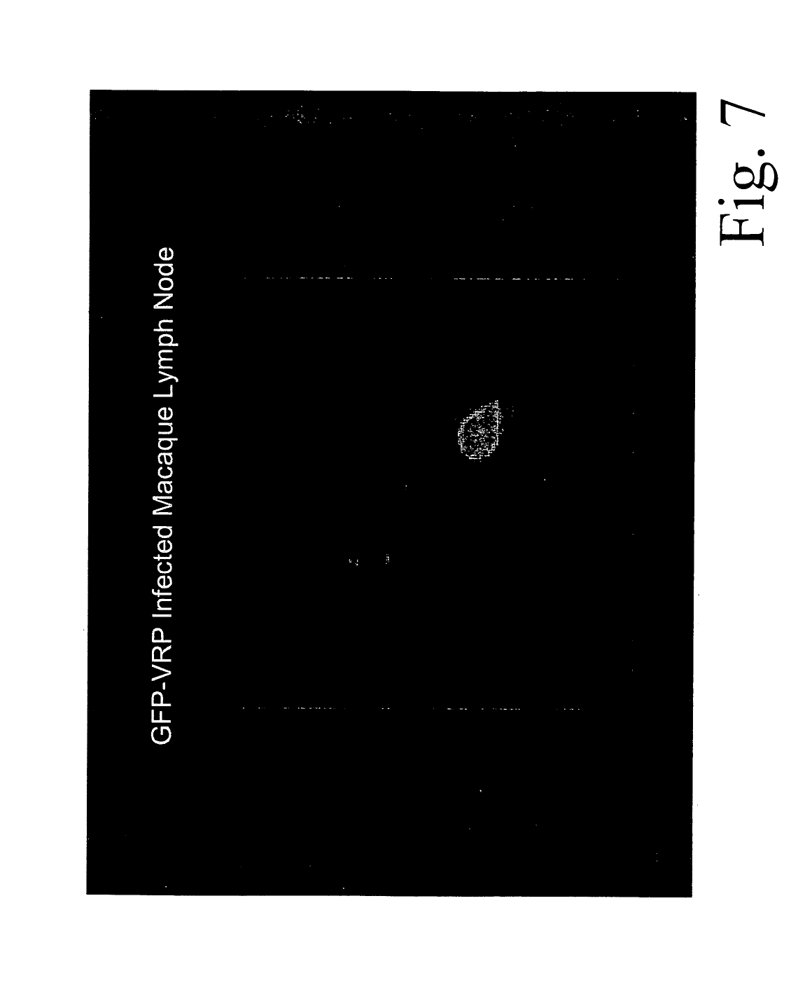

GFP-VRP-3000 (10⁴ IU VRP in 0.5 ml PBS) was administered to four rhesus macaques, 5 cm lateral to the inguinal triangle as described in Example 6. Eighteen hours post-inoculation, simple excision of the inguinal lymph nodes were performed and processed for fluorescence microscopy as described in Example 1 (FIG. 7). The positive fluorescence observed indicates that dendritic cells are targeted by wild-type GFP-VRP in macaques.

### EXAMPLE 11

### Heparin Affinity Chromatography of Mutagenized Viral Particles

Heparin affinity chromatography can be performed using any of several commercially available resins to which heparin has been bound. The source of heparin in these columns is variable; current commercially available resins use porcine heparin, but other sources can be used effectively.

A. Pharmacia HiTrap® Heparin

Columns of Pharmacia HiTrap® Heparin (cat no. 17-0407-01, Amersham Pharmacia Biotech) are pre-equilibrated with 25 mM HEPES/0.25 M NaCl, pH 7.5, and then loaded with mutagenized virus preparations as described above. Non or weakly binding mutants are collected in the first eluants from the column, i.e. where the non-bound materials elute.

B. Heparin Sepharose 6 Fast Flow® resin

Heparin Sepharose 6 Fast Flow® resin (catalog no. 90-1000-2; Amersham Pharmacia Biotech) is supplied as a bulk resin which allows various size columns to be packed as needed. A 6 ml column is prepared by packing the Heparin Sepharose 6 Fast Flow® bulk resin in a BioRad® Econo-Column chromatography column, then pre-equilibrated with 25 mM HEPES/0.12 M NaCl, pH 7.5. Mutagenized viral preparations are loaded onto the column, and non- or weakly binding mutants are collected in the first eluants from the column.

The foregoing is illustrative of the present invention, and is not to be construed as limiting thereof. The invention is defined by the following claims, with equivalents of the claims to be included therein.

1. A composition comprising a population of infectious, attenuated, alphavirus replicon particles in an immunogenically effective dosage, wherein each of said alphavirus particles comprises:
(a) a virion shell comprising Venezuelan Equine Encephalitis (VEE) structural proteins, wherein said virion shell further comprises an attenuating mutation in the E1 glycoprotein;
(b) a recombinant alphavirus replicon RNA comprising a heterologous nucleotide sequence encoding an immunogen, wherein said heterologous nucleotide sequence is operably associated with a promoter,
wherein said immunogenically effective dosage comprises a number of infectious alphavirus particles that is (i) substantially the same as or substantially less than the immunogenically effective dosage of a comparable alphavirus having a wild-type VEE virion shell or (ii) is less than about 1 00-fold more than the immunogenically effective dosage of a comparable alphavirus having a wild-type VEE virion shell.

2. The composition of claim 1, wherein said immunogenically effective dosage comprises substantially the same number of infectious alphavirus particles as an immunogenically effective dosage of a comparable virus having a wild-type VEE virion shell.

3. The composition of claim 1, wherein said immunogenically effective dosage comprises a substantially lower number of infectious alphavirus particles than an immunogenically effective dosage of a comparable alphavirus having a wild-type VEE virion shell.

4. A composition comprising a population of infectious, attenuated, alphavirus replicon particles in an immunogenically effective dosage, wherein each of said alphavirus particles comprises:
(a) a virion shell comprising Venezuelan Equine Encephalitis (VEE) structural proteins, wherein said virion shell further comprises an attenuating mutation in the E1 glycoprotein;
(b) a recombinant alphavirus replicon RNA comprising a heterologous nucleotide sequence encoding an immunogen,
wherein said alphavirus particles exhibit only weak or no detectable binding to heparin.

5. The composition of claim 1, wherein said attenuating mutation in the E1 glycoprotein comprises an attenuating mutation in the fusogenic peptide region.

6. The composition of claim 1, wherein said attenuating mutation in the E1 glycoprotein comprises an attenuating mutation selected from the group consisting of (i) an attenuating mutation at E1 glycoprotein amino acid position 81, and (ii) an attenuating mutation at E1 glycoprotein amino acid position 253.

7. The composition of claim 6, wherein said VEE virion shell comprises a Phe→Ile attenuating mutation at E1 glycoprotein amino acid position 81.

8. The composition of claim 6, wherein said VEE virion shell comprises a Phe→Ser attenuating mutation at E1 glycoprotein amino acid position 253.

9. The composition of claim 1, wherein said composition comprises about 10² to about 10⁶ infectious alphavirus particles.

10. The composition of claim 1, wherein said composition comprises about 10³ to about 10⁵ infectious alphavirus particles.

11. The composition of claim 1, wherein said composition comprises about 10⁵ to about 10⁹ infectious alphavirus particles.

12. The composition of claim 11, wherein said composition comprises about 10⁶ to about 10⁸ infectious alphavirus particles.

13. The composition of claim 1, wherein said recombinant alphavirus replicon RNA is a recombinant VEE replicon RNA.

14. The composition of claim 1, wherein said promoter is an alphavirus 26S subgenomic promoter.

15. The composition of claim 1, wherein said immunogen is a cancer immunogen.

16. The composition of claim 1, wherein said immunogen is an infectious disease immunogen.

17. The composition of claim 16, wherein said immunogen is selected from the group consisting of a bacterial immunogen, a viral immunogen, and a protozoa immunogen.

18. The composition of claim 1, wherein said immunogen is a Simian Immunodeficiency Virus (SIV) immunogen or a Human Immunodeficiency Virus (HIV) immunogen.

19. The composition of claim 18, wherein said immunogen is a SIV or HIV immunogen selected from the group consisting of a gag, env, ref, tat, nef and pol gene product, and a combination thereof.

20. The composition of claim 1, wherein said replicon RNA lacks sequences encoding the VEE structural proteins.

21. A pharmaceutical formulation comprising the composition of claim 1 in a pharmaceutically acceptable carrier.

22. A method of producing an immune response in a subject, comprising administering to the subject an immunogenically effective amount of a composition according to claim 1.

23. A method of producing an immune response in a subject, comprising:
(a) administering ex vivo to a plurality of cells a composition according to claim 1, and
(b) administering an immunogenically effective amount of the cells to the subject.

24. The method of claim 23, wherein the plurality of cells comprises dendritic cells.

25. The method of claim 22, wherein a protective immune response is induced in the subject.

26. The method of claim 22, wherein said administering step is carried out by subcutaneous administration.

27. The method of claim 22, wherein said administering step is carried out by intradermal administration.

28. The method of claim 22, wherein said administering step is carried out by administration to a limb of the subject.

29. The method of claim 28, wherein said administering step is to a front limb of the subject.

30. The method of claim 22, wherein the subject is a mammalian subject.

31. The method of claim 30, wherein the subject is selected from the group consisting of a primate subject, a pig, a cow, a dog and a cat.

32. The method of claim 31, wherein the subject is a human subject.

33. The method of claim 32, wherein the subject has, or is at risk of developing, AIDS.

34. The method of claim 24, wherein the heterologous nucleotide sequence is introduced into the dendritic cells and the dendritic cells express the immunogen.

35. The method of claim 24, wherein said contacting step is carried out in vitro.

36. The method according to claim 24, wherein said contacting step is carried out in vivo.
